(19) United States
(12) Patent Application Publication
(10) Pub. No.: US 2008/0075768 A1
(43) Pub. Date: Mar. 27, 2008

(54) HYDROPHOBIC OPIOID ABUSE DETERRENT DELIVERY SYSTEM USING OPIOID ANTAGONISTS

(76) Inventors: Jason M. Vaughn, Round Rock, TX (US); Michael M. Crowley, Austin, TX (US); Feng Zhang, Austin, TX (US); John J. Koleng, Austin, TX (US); Justin M. Keen, Round Rock, TX (US); Justin R. Hughey, Austin, TX (US)

Correspondence Address: MEYERTONS, HOOD, KIVLIN, KOWERT & GOETZEL, P.C., P.O. BOX 398, AUSTIN, TX, 78767-0398, US

(21) Appl. No.: 11/781,044

(22) Filed: Jul. 20, 2007

Related U.S. Application Data
(60) Provisional application No. 60/820,091, filed on Jul. 21, 2006.
(60) Provisional application No. 60/824,042, filed on Aug. 30, 2006.
(60) Provisional application No. 60/871,504, filed on Dec. 22, 2006.
(60) Provisional application No. 60/824,057, filed on Aug. 30, 2006.
(60) Provisional application No. 60/903,235, filed on Feb. 22, 2007.
(60) Provisional application No. 60/893,825, filed on Mar. 8, 2007.
(60) Provisional application No. 60/893,798, filed on Mar. 8, 2007.

(51) Int. Cl. A61K 9/48; A61K 47/12; A61K 47/38; A61P 25/36; A61K 9/00

(52) U.S. Cl. 424/456; 424/484; 514/781; 514/784

(57) ABSTRACT

Disclosed herein are oral dosage forms of opioid therapeutic agents that are resistant to abuse and methods of their formulation. In particular, oral dosage forms that are resistant to dissolution in aqueous solutions of ethanol are described. The oral dosage forms may include one or more opioid antagonists that are sequestered from the opioid therapeutic agent such that the opioid antagonist has no substantial effect on the activity of the opioid therapeutic agent when the dosage form is taken orally as prescribed, but the opioid antagonist is released in an amount that reduces the effectiveness of the opioid therapeutic agent contained in the dosage form when the dosage form is crushed.

This application claims the benefit of U.S. Provisional Application No. 60/820,091 entitled “Abuse Deterrent Delivery System,” filed Jul. 21, 2006 and U.S. Provisional Application No. 60/824,042 entitled “Hydrophobic Abuse Deterrent Delivery System,” filed Aug. 30, 2006 and U.S. Provisional Application No. 60/871,504 entitled “Hydrophobic Abuse Deterrent Delivery System,” filed Dec. 2, 2006 and U.S. Provisional Application No. 60/824,057 entitled “Hydrophilic Abuse Deterrent Delivery System” filed Aug. 30, 2006 and U.S. Provisional Application No. 60/903,235 entitled “Hydrophilic Abuse Deterrent Delivery System” filed Feb. 22, 2007 and U.S. Provisional Application No. 60/893,825 entitled “Hydrophobic Abuse Deterrent Delivery System For Opioid Agents” filed Mar. 8, 2007 and U.S. Provisional Application No. 60/893,798 entitled “Hydrophilic Abuse Deterrent Delivery System For Opioid Agents” filed Mar. 8, 2007.

### BACKGROUND OF THE INVENTION

1. Field of the Invention

The invention generally relates to pharmaceutical delivery systems and methods of their use, in particular oral dosage systems for the delivery of drugs that are resistant to abuse.

2. Description of the Relevant Art

Drug formulations for the oral delivery of pharmaceuticals have been used for centuries. More recently, numerous compositions and methods have been developed for the controlled release of pharmaceuticals after oral delivery. Such extended-release characteristics can be useful for many reasons. One reason is that extended-release delivery systems can limit the number of doses a patient must take over a period of time thus improving compliance with a dosing regimen. Another reason is that extended release delivery systems can provide a steady dose of medication to a patient, thereby avoiding sudden increases and decreases in the level of medication being delivered to the bloodstream. Controlled release of pharmaceuticals is particularly critical with drugs that are habit forming, as the controlled release of the medication can significantly reduce the likelihood of a patient developing an addiction to the substance.

One common method of producing a controlled release oral dosage form is to surround the drug with a coating or barrier of a hydrophobic substance such as a polymeric coating. These coatings or barriers can be designed to dissolve gradually when brought into contact with digestive fluids thus producing a slow and steady release of a drug when it is ingested.

Other approaches that have been developed include the methods disclosed in U.S. Pat. Nos. 6,261,599, 6,335,033, 6,706,281 and 6,743,442 wherein a drug is mixed with a water-insoluble retardant and optionally with binders and/or plasticizers. The mixture is then heated and extruded into narrow strands which are cut into particles having a size of about 0.1 to about 12 mm in length and a diameter from about 0.1 to about 5 mm. The particles may then be incorporated into a capsule that delivers a suitable dose of the therapeutic agent.

The difficulty in the art is that it is desirable among drug abusers to bypass the extended release characteristics of oral dosage forms. By negating the controlled release mechanisms of the dosage form, the abuser is able to produce a quick and intense rush of drug into the brain that results in a high. Abusers have found many methods by which the extended release characteristics of certain oral dosage forms can be bypassed. These include: (i) intravenous injection of dissolved tablets or capsules, (ii) inhalation/nasal snorting of crushed tablets or capsules, (iii) chewing tablets or capsules and (iv) dissolving of tablets or capsules in alcoholic beverages followed by oral administration.

Abuse of narcotic substances is particularly problematic. Such drugs are highly habit forming when misused and thus are in high demand by drug abusers. In contrast, there are numerous legitimate users of narcotic substances that need oral dosage forms that release large quantities of narcotic over an extended period of time for the treatment of extreme pain.

Oral formulations that deter abuse have also been suggested. U.S. Pats. No. 5,747,058 and 5,968,542 and U.S. Publication No. 200401611382 disclose an oral drug delivery system based on the use of therapeutic agents suspended in high viscosity liquid carrier material.

The U.S. Publication No. 20030118641 discloses controlled-release opioid delivery compositions that are resistant to extraction with commonly-available solvents. The formulation between 30 and 65% of a matrix forming polymer and between 5 and 15% of an ionic exchange resin. However the disclosed formulations are prepared as tablets of compressed powder that can be readily crushed. This fails to deter methods of drug abuse involving nasal inhalation.

Other abuse deterrent systems include oral dosage forms that include an opioid and an opioid antagonist that is released when the dosage form is tampered with. Examples of this approach can be found at U.S. Pat. Nos. 6,696,088, 6,696,066, 6,627,635, 6,326,027 and 6,228,863.

U.S. Publication No. 20040052731 discloses oral dosage forms of drugs that have been modified to increase their lipophilicity entrapped in coated microparticles wherein the coatings render the microparticles insoluble or poorly soluble in various solvents. The formulations can still be crushed, but the formulations are intended to prevent immediate release of the drug even when crushed.

Therefore there remains a significant need in the art for oral dosage forms that are resistant to attempts by potential abusers to bypass the controlled or extended release characteristics of conventional oral dosage forms. In particular, oral dosage forms are needed that are resistant to crushing and dissolution in water or aqueous alcohol solutions such as alcoholic beverages.

### SUMMARY OF THE INVENTION

In certain embodiments, the invention relates to oral dosage forms that are resistant to ethanol extraction or dose-dumping in ethanol, preferably wherein the oral dosage form releases less than 40% of the therapeutic agent after 5 minutes of shaking at 240 cycles/min in a 0.1 N HCl solution followed by 3 hours of shaking on an orbital shaker at 240 cycles/min in an acidic aqueous solution of 40% ethanol at 25° C. The disclosed formulations are also resistant to opioid abuse by including a therapeutic amount of an opioid agent and an effective amount of an opioid antagonist. The opioid antagonist is sequestered from the opioid therapeutic agent such that the opioid antagonist has no significant effect on the activity of the opioid therapeutic agent when the dosage form is taken orally as prescribed. Tampering with the dosage form, or crushing the dosage form however, releases the opioid antagonist in an amount effect to reduce the abuse potential of the opioid therapeutic agent.

In certain embodiments, the invention relates to oral dosage forms of an opioid therapeutic agent that are abuse deterrent. In one embodiment, a monolithic solidified oral dosage form is described which is prepared by a thermal process. The oral dosage form comprises an opioid therapeutic agent, an opioid antagonist, and a hydrophobic matrix material. The oral dosage form releases at least 80% of the therapeutic agent after 2 hours of stirring in a 0.1 N HCl solution and 16 hours stirring in a pH 6.8 phosphate buffer solution using a United States Pharmacopoeia (USP) Type II paddle apparatus at 75 rpm and 37° C. Additionally, the oral, dosage form exhibits abuse deterrent properties. For example, the oral dosage form releases less than 40% of the therapeutic agent after 5 minutes of shaking at 240 cycles/min in a 0.1 N HCl solution followed by 3 hours of shaking on an orbital shaker at 240 cycles/min in an acidic aqueous solution of 40% ethanol at 25° C. The opioid antagonist is sequestered from the opioid therapeutic agent such that the opioid antagonist has no significant effect on the activity of the opioid therapeutic agent when the dosage form is taken orally as prescribed. Tampering with the dosage form, or crushing the dosage form however, releases the opioid antagonist in an amount effect to reduce the abuse potential of the opioid therapeutic agent.

The oral dosage forms can further comprise one or more plasticizers, emetics, nasal irritants or functional excipients such as colorants, lubricants, thermal lubricants, antioxidants, buffering agents, disintegrants, binders, diluents, sweeteners, chelating agents, flavorants, surfactants, solubilizers, stabilizers, hydrophilic polymers, hydrophobic polymers, waxes, lipophilic materials, absorption enhancers, preservative, absorbent, cross-linking agents, bioadhesive polymers, pore formers, osmotic agents, polycarboxylic acids, and fragrance, or combinations thereof.

The invention further relates to methods of formulating an oral dosage form that deters abuse. The oral dosage form may be made by: mixing one or more water-insoluble polymers, an opioid therapeutic agent, and an opioid antagonist, wherein the water-insoluble polymers comprises 20 to 99.9% of the mixture by weight; melting the mixture; and permitting the mixture to solidify as a substantially solid oral dosage form, wherein the oral dosage form weighs at least 40 mg. The opioid antagonist is sequestered from the opioid therapeutic agent such that the opioid antagonist has no significant effect on the activity of the opioid therapeutic agent when the dosage form is taken orally as prescribed. Tampering with the dosage form, or crushing the dosage form however, releases the opioid antagonist in an amount effect to reduce the abuse potential of the opioid therapeutic agent.

In yet other embodiments, a method of providing a therapeutic agent to a patient includes providing a monolithic solidified oral dosage form which is prepared by a thermal process. The oral dosage form comprises an opioid therapeutic agent, an opioid antagonist, and a hydrophobic matrix material. The oral dosage form releases at least 80% of the therapeutic agent after 2 hours of stirring in a 0.1 N HCl solution and 16 hours stirring in a pH 6.8 phosphate buffer solution using a USP Type II paddle apparatus at 75 rpm and 37° C. Additionally, the oral dosage form exhibits abuse deterrent properties. For example, the oral dosage form releases less than 40% of the therapeutic agent after 5 minutes of shaking at 240 cycles/min in a 0.1 N HCl solution followed by 3 hours of shaking on an orbital shaker at 240 cycles/min in an acidic aqueous solution of 40% ethanol at 25° C. The opioid antagonist is sequestered from the opioid therapeutic agent such that the opioid antagonist has no significant effect on the activity of the opioid therapeutic agent when the dosage form is taken orally as prescribed. Tampering with the dosage form, or crushing the dosage form however, releases the opioid antagonist in an amount effect to reduce the abuse potential of the opioid therapeutic agent.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1: Chart depicting the release over time of metronidazole from an oral dosage form when shaken in acidified aqueous ethanol solution;

FIGS. 2A and 2B depict the release over time of hydromorphone from an oral dosage form described in Example 3 when stirred in an aqueous solution;

FIG. 3 depicts the release over time of hydromorphone from an oral dosage form described in Example 3 when shaken in an aqueous ethanol solution;

FIGS. 4A and 4B depict the release over time of hydromorphone from an oral dosage form described in Example 4 when stirred in an aqueous solution;

FIG. 5 depicts the release over time of hydromorphone from an oral dosage form described in Example 4 when shaken in an aqueous ethanol solution;

FIGS. 6A and 6B depict the release over time of hydromorphone from an oral dosage form described in Example 5 when stirred in an aqueous solution;

FIG. 7 depicts the release over time of hydromorphone from an oral dosage form described in Example 5 when shaken in an aqueous ethanol solution;

FIG. 8 depicts the release over time of hydromorphone from an oral dosage form described in Example 6 when stirred in an aqueous solution;

FIG. 9 depicts the release over time of hydromorphone from an oral dosage form described in Example 7 when stirred in an aqueous solution;

FIG. 10 depicts the release over time of hydromorphone from an oral dosage form described in Example 7 when shaken in acidified aqueous ethanol solution;

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

Embodiments described herein relate to oral dosage forms that are designed to deter misuse of controlled substances or other therapeutic agents. Furthermore, the embodiments described herein are directed to methods of formulating such oral dosage forms. Additionally, embodiments described herein provide methods of deterring substance abuse. As used herein, “abuse deterrent” oral dosage forms exhibit the following properties: (i) are resistant to dissolution in water, thus inhibiting intravenous injection of dissolved oral dosage form; (ii) are resistant to breaking thus inhibiting abuse by inhalation/nasal snorting of crushed tablets or capsules or by chewing tablets or capsules and (iii) are resistant to dissolution in aqueous ethanolic solutions or pure ethanol, thus inhibiting oral administration by dissolving in alcoholic beverages.

In one embodiment, oral dosage forms are provided that are significantly harder than conventional oral dosage forms and which are relatively insoluble in water, aqueous solutions of 40% ethanol, or acidified aqueous solutions of 40% ethanol.

Hardness of the oral dosage form presents a significant deterrent to abuse because the dosage forms cannot be readily crushed for inhalation, or dissolution prior to oral ingestion or intravenous use. They are also resistant to being crushed by chewing. Indeed, in certain embodiments the oral dosage forms are so hard that tablets made according to the embodiments described herein may be pounded with a hammer and still incur surprisingly little damage. Crushing oral dosage forms described in embodiments disclosed herein would pose a significant challenge to a potential abuser.

The relative insolubility of the oral dosage forms in water or aqueous solutions of 40% ethanol is a deterrent to abuse because it is difficult and time-consuming to prepare the dosage form for oral ingestion. In the case of many of the oral dosage forms disclosed herein, not only is dissolution of the oral dosage form for intravenous injection difficult, the resulting solution would contain water-insoluble polymers that could cause serious internal damage if injected intravenously in significant quantities.

In preferred embodiments the oral dosage form is monolithic and substantially solid, that is it is formed as a unitary mass that is molded, cut, ground or otherwise formed in its final shape, and is not, for example, an aggregate or composite of individual solid particulates, pellets, beads microspheres or the like. Preferably, the monolithic substantially solid oral dosage form is formed by providing a mixture including a suitable thermoplastic polymeric retardant (e.g., a hydrophobic polymer) and a therapeutic agent, melting the mixture and permitting the mixture to solidify as a substantially solid oral dosage form. Embodiments described herein further provide methods of administering a therapeutic agent to a patient that include supplying said substantially solid oral dosage form to a patient.

The phrase “oral dosage form” as used herein refers to pharmaceutical compositions formed as tablets, caplets and the like that are swallowed substantially intact when used as intended. Films, wafers and the like which are not intended to be swallowed substantially intact are not contemplated embodiments of oral dosage forms.

The hardness of an oral dosage form can be determined using a standard test known to those of skill in the art. That test is called Hardness or Crushing Strength and it involves the following steps: a dosage form is compressed between a moving piston and a stationary plate until it laminates, ruptures or breaks. The force required to laminate, rupture or break the dosage form is a measure of its hardness or breaking strength. Typical solid oral dosage forms exhibit hardness values between 4-18 kp. In contrast to conventional oral dosage forms, the oral dosage forms of the described embodiments have a hardness at room temperature of at least about 20 kp, at least about 30 kp, at least about 35 kp, at least about 40 kp, or at least about 50 kp.

The solubility of oral dosage forms in aqueous solutions of 40% ethanol (a standard test widely used in the art) may be determined by placing the oral dosage form in a room-temperature aqueous solution of 40% ethanol and stirring or shaking the solution for a period of time. In one typical method, the oral dosage form in 60 mL of an aqueous solution of 40% ethanol is shaken for 3 hours in an orbital shaker at 240 cycles/min. Preferably, the volume of 40% ethanol used is 60 mL, or approximately 2 fluid ounces. In some instances, acidified aqueous solutions of 40% ethanol are used, particularly when the oral dosage form is disposed in a gelatin-capsule or coated with a gelatin coating, which are otherwise insoluble in 40% ethanol. In one embodiment, the oral dosage form releases less than 40% of the hydromorphone and/or pharmaceutically acceptable salts of hydromorphone after 5 minutes of shaking at 240 cycles/min in a 0.1 N HCl solution, to at least partially dissolve the capsule material or remove a coating material, followed: by 3 hours of shaking on an orbital shaker at 240 cycles/min in an acidic aqueous solution of 40% ethanol at 25° C. Different shaking methods and alternate periods of time can be used, if appropriate, and such variations would be well-known to those skilled in the art. However for the purposes of this disclosure the typical method described above was used to determine the solubility of the oral dosage forms. For the purposes of this disclosure, an oral dosage form is insoluble in a 40% solution of aqueous ethanol if three hours of shaking according to the protocol described above results in a release of less than about 40% of the therapeutic agent, preferably less than about 30% of the therapeutic agent, more preferably less than about 20% of the therapeutic agent and most preferably less than about 10% of the therapeutic agent.

Thermoplastic Polymeric Retardant

In certain embodiments, an oral dosage form includes a polymeric retardant in which one or more therapeutic agents are suspended. In an embodiment, the polymeric retardant is a fusible, thermoplastic or thermosetting material, typically a resin or polymer.

In some embodiment, a thermoplastic polymeric retardant is a hydrophobic matrix material. The hydrophobic matrix material, in some embodiments, is a pharmaceutically acceptable carrier and preferably is (i) capable of producing an oral dosage form that has a hardness of at least about 20 kp, 25 kp, 30 kp, 35 kp, 40 kp or 50 kp and additionally or alternatively (ii) releases less than about 40%, less than about 30%, less than about 20% or less than about 10% of a therapeutic agent when subjected to shaking in aqueous ethanol solution as described above.

For purposes of the present disclosure a matrix material is considered to be hydrophobic or water-insoluble if it is “sparingly soluble” or “practically insoluble” or “insoluble” as defined by USP 29/NF 24. However the hydrophobic matrix also preferably has physical characteristics that produce a suitable level of release of the therapeutic agent within the gastrointestinal tract. In other preferred embodiments the hydrophobic material is soluble or slightly soluble in aqueous solution at a pH of at least about 5.5 or greater. Most preferably, the hydrophobic polymer is soluble or slightly soluble in intestinal fluid but is not soluble in gastric fluid.

The release characteristics of the oral dosage form can be determined in vitro using simulated gastric or intestinal fluids, but is preferably determined in vivo by monitoring blood levels of the therapeutic agent in subjects that have ingested the oral dosage form. Methods of determining the in vivo and in vitro release of therapeutic agents from oral dosage forms are well-known to those skilled in the art. Extended release oral dosage forms will typically result in an therapeutically-acceptable, extended-time release of therapeutic agents over a period of at least about 4, 6, 8, 10, 12, 14, 16, 18, 20, 22, 24, 30, 36, 48, 60 or 72 hours.

In preferred embodiments, the hydrophobic matrix material may be one or more water insoluble polymers. A single water-insoluble polymer or a mixture of water-insoluble polymers can be used to make up the hydrophobic matrix of the oral dosage form. When used as the hydrophobic matrix material the water-insoluble polymer or polymers preferably include about 20% to about 99.9% of the oral dosage form by weight, more preferably at least about 30%, more preferably about 40% and most preferably at least about 50% of the oral dosage form by weight

In some embodiments, the hydrophobic matrix material is a pharmaceutically-acceptable, water-insoluble polymer (i.e., a hydrophobic polymer). Examples of pharmaceutically-acceptable, water-insoluble polymers include, but are not limited to acrylic acid-based polymers, methacrylic acid based polymers, and acrylic acid—methacrylic acid based copolymers. As used herein, the phrase “acrylic acid-based polymers” refers to any polymer that includes one or more repeating units that include and/or are derived from acrylic acid. As used herein, the phrase “methacrylic acid-based polymers” refers to any polymer that includes one or more repeating units that include and/or are derived from methacrylic acid. Derivatives of acrylic acid and methacrylic acid include, but are not limited to, alkyl ester derivatives, alkylether ester derivatives, amide derivatives, alkyl amine derivatives, anhydride derivatives, cyanoalkyl derivatives, and amino-acid derivatives. Examples of acrylic acid-based polymers, methacrylic acid based polymers, and acrylic acid—methacrylic acid based copolymers include, but are nor limited to Eudragit® L100, Eudragit® L100-55, Eudragit® L 30 D-55, Eudragit® S100, Eudragit® 4135F, Eudragit® RS, acrylic acid and methacrylic acid copolymers, methyl methacrylate polymers, methyl methacrylate copolymers, polyethoxyethyl methacrylate, polycyanoethyl methacrylate, aminoalkyl methacrylate copolymer, polyacrylic acid, polymethacrylic acid, methacrylic acid alkylamine copolymer, polymethyl methacrylate, polymethacrylic acid anhydride, polyalkylmethacrylate, polyacrylamide, and polymethacrylic acid anhydride and glycidyl methacrylate copolymers.

Further examples of pharmaceutically-acceptable water-insoluble polymers include, but are not limited to, alkylcelluloses such as ethylcellulose, methylcellulose, calcium carboxymethyl cellulose, certain substituted cellulose polymers such as hydroxypropyl methylcellulose phthalate, and hydroxypropyl methylcellulose acetate succinate, cellulose acetate butyrate, cellulose acetate phthalate, and cellulose acetate trimaleate, polyvinyl acetate phthalate, polyvinyl acetate, polyester, waxes, shellac, zein, or the like.

In further embodiments, in addition to containing 20 to 99.9% by weight of one or more pharmaceutically-acceptable, hydrophobic matrix materials, the oral dosage forms may further include one or more pharmaceutically-acceptable hydrophilic matrix materials including water-soluble polymers such as polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, carboxymethyl cellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin or the like.

For purposes of the present disclosure, a matrix material is considered hydrophilic and a polymer is considered to be water-soluble if it is more than sparingly soluble as defined by USP 29/NF 24, that is if according to USP 29/NF 24 the matrix material or polymer is classified as “soluble” or “very soluble.”

Preferred materials used to produce an oral dosage form will be pharmaceutically acceptable materials, such as those indicated to be generally regarded as safe (“GRAS-certified”) or national formulary certified.

Therapeutic Agents

Oral dosage forms also include a therapeutic agent. In preferred embodiments the therapeutic agent is a drug that has a potential for abuse. The United States Drug Enforcement Administration makes determinations about various therapeutic a potential for abuse and assigns them to various schedules. Schedule I drugs or other substances are compounds with a high potential for abuse which currently have no accepted medical uses for treatment in the United States, in some instances due to the extremely high potential for abuse. Schedule II drugs or other substances are compounds with a high potential for abuse and which have medically acceptable uses in the United States when used under severe restrictions. When abused schedule II drugs may lead to severe psychological or physical dependence in a user. Schedule III drugs are drugs that have some potential for abuse and that have a currently accepted medical use in the United States. Abuse of schedule II drugs or substances may lead to moderate to low physical dependence or high psychological dependence. Schedule IV and schedule V drugs or substances have a low potential for abuse and abuse of these compounds leads to more limited or non-existent physical or psychological dependence.

The compositions and methods disclosed herein will most preferably be used with therapeutic agents that are or have been designated as schedule II or schedule III drugs or substances. The compositions and methods disclosed herein may also be used to develop medically-acceptable oral dosage forms of therapeutic agents that are designated as schedule I drugs or substances. In other embodiments, it may also be desirable to formulate therapeutic agents that are designated as schedule IV or schedule V drugs or substances according to the compositions and methods disclosed herein to prevent abuse.

In preferred embodiments, the therapeutic agent will be a narcotic. The narcotic can be an opioid such as alfentanil, allylprodine, alphapro dine, anileridine, apomorphine, apo codeine, benzylmorphine, bezitramide, buprenorphine, butorphanol, clonitazene codeine, codeine methyl bromide, codeine phosphate, codeine sulfate, cyclazocine, cyclorphen, cyprenorphine, desomorphine, dextromoramide, dezocine, diampromide, dihydrocodeine, dihydrocodeinone enol acetate, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydrocodone bitartrate, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, morphine derivatives, myrophine, nalbuphine, narceine, nicomorphine, norlevorphanol, normethadone, nalorphine, normorphine, norpipanone, ohmefentanyl, opium, oxycodone, oxymorphone, papavereturn, pentazocine, phenadoxone, phenomorphan, phenazocine, phenoperidine, pheoperidine, pholcodine, piminodine, piritramide, propheptazine, promedol, profadol, properidine, propiram, propoxyphene, remifentanyl, sufentanyl, tramadol, tilidine, naltrexone, naloxone, nalmefene, methylnaltrexone, naloxone methiodide, naloxonazine, nalide, nalmexone, nalbuphine, nalorphine dinicotinate, naltrindole (NTI), naltrindole isothiocyanate, (NTII), naltriben (NTB), norbinaltorphimine (nor-BNI), β-funaltrexamine (b-FNA), BNTX, cyprodime, ICI-174,864, LY117413, MR2266, etorphine, DAMGO, CTOP, diprenorphine, naloxone benzoylhydrazone, bremazocine, ethylketocyclazocine, U50,488, U69,593, spiradoline, DPDPE, [D-Ala2,Glu4] deltorphin, DSLET, Metenkephalin, Leu-enkephalin, β-endorphin, dynorphin A, dynorphin B, a-neoendorphin, or an opioid having the same pentacyclic nucleus as nalmefene, naltrexone, buprenorphine, levorphanol meptazinol, pentazocine, dezocine, or the pharmacologically effective esters or salts of any of the foregoing opioids.

In other embodiments the therapeutic agent will be a CNS depressant, sedative or hypnotic such as acyclic ureides such as Acecarbromal, Apronalide, Bomisovalum, Capuride, Carbromal and Ectylurea; alcohols such as Chlorhexadol, Ethchlorvynol, Meparfynol, 4-Methyl-5-thiazoleethanol, tert-Pentyl Alcohol and 2,2,2-Trichloroethanol; amides such as Butoctamide, Diethylbromoacetamide, Ibrotamide, Isovaleryl Diethylamide, Niaprazine, Tricetamide, Trimetozine, Zolpidem and Zopiclone; barbituric acid derivatives such as Allobarbital, Amobarbital, Aprobarbital, Barbital, Brallabarbital, Butabarbital Sodium, Butalbital, Butallylonal, Butethal, Carbubarb, Cyclobarbital, Cyclopentobarbital, Enallylpropymal, 5-Ethyl-5-(1-piperidyl)barbituric Acid, 5-Furfuryl-5-isopropylbarbituric Acid, Heptabarbital Hexethal Sodium, Hexobarbital, Mephobarbital, Methitural, Narcobarbital, Nealbarbital, Pentobarbital Sodium, Phenallymal, Phenobarbital, Phenobarbital Sodium, Phenylmethylbarbituric Acid, Probarbital, Propallylonal, Proxibarbal, Reposal, Secobarbital Sodium, Thiopental, Talbutal, Tetrabarbital, Thiobarbital, Thiamylal, Vinbarbital Sodium and Vinylbital; Benzodiazepine derivatives such as alprazolam, Brotizolam, clorazepate, chlordiazepoxide, clonazepam, diazepam, Doxefazepam, Estazolam, Flunitrazepam, Flurazepam, Haloxazolam, lorazepam, Loprazolam, Lormetazepam, Nitrazepam, Quazepam, Temazepam and Triazolam; bromides such as Ammonium Bromide, Calcium Bromide, Calcium Bromolactobionate, Lithium Bromide, Magnesium Bromide, Potassium Bromide and Sodium Bromide; carbamates such as Amyl. Carbamate-Tertiary, Ethinamate, Hexaprpymate, Meparfynol Carbamate, Novonal and Tricholorourethan; chloral derivatives such as Carbocloral, Chloral Betaine, Chloral Formamide, Chloral Hydrate, Chloralantipyrine, Dichloralphenazone, Pentaerythritol Chloral and Triclofos; piperidinediones such as Glutehimide, Methyprylon, Piperidione, Pyrithyldione, Taglutimide and Thalidomide; quinazolone derivatives such as Etaqualone, Mecloqualone and Methaqualone; and others such as Acetal, Acetophenone, Aldol, Ammonium Valerate, Amphenidone, d-Bornyl a-Bromoisovalerate, d-Bomyl Isovalerate, Bromoform, Calcium 2-Ethylbutanoate, Carfinate, a-Chlorolose, Clomethiazole, Cypripedium, Doxylamine, Etodroxizine, Etomidate, Fenadiazole, Homofenazine, Hydrobromic Acid, Mecloxamine, Menthyl Valerate, Opium, Paraldehyde, Perlapine, Propiomazine, Rilmazafone, Sodium Oxybate, Sulfonethylmethane and Sulfonmethane.

In yet other embodiments the therapeutic agent can be any suitable therapeutic agent, and preferably those subject to abuse, including but not limited to the following: (A) stimulants, for example amphetamine (including dextroamphetamine and levoamphetamine), methamphetamine, methylphenidate (Ritalin®), phenmetrazine, modatinil, advafinil, armodafinil, and ampakimes such as CX516, CX546, CX614, and CX717.

(B) cannabinoids such as tetrahydro-cannabinol, nabilone, hashish and hashish oil and 1-piperidinocyclohexanecarbonitrile;

(C) dissociatives such as phencyclidine (PCP), ketamine, tiletamine, dextromethorphan, ibogaine, dixocilpine and riluzole;
(D) steroid or hormonal active agent (including both natural, semi-synthetic and synthetic compounds and their derivatives having steroidal or hormonal activity) including, for example, (a) estrogens such as Colpormon, Conjugated Estrogens, Estradiol (17β- and α-) and its Esters (e.g., Acetate, Benzoate, Cypionate, Dipropionate Diacetate, Enanthate, Estradiol-16,17-Hemisuccinate, Undececenoate, Undecylate and Valerate), Estriol, Estrone, Ethinyl Estradiol, Equilenin, Equilin, Mestranol, Methyl Estradiol, Moxestrol, Mytatrienediol, Quinestradiol, Quinestrol, Dienestrol, Clomifen, Chlorotrianisen, and Cyclofenil; (b) progestagenically effective hormones such as Allylestrenol, Anagestone, Chlormadinone Acetate, Delmadinone Acetate, Demegestone, Desogestrel, 3-Keto Desogestrel, Dimethisterone, Dydrogesterone, Ethinylestrenol, Ethisterone, Ethynodiol (and Diacetate), Fluorogestone Acetate, Gestodene, Gestonorone Caproate, Haloprogesterone, (17-Hydroxy- and 17-Acetate-) 16-Methylene-Progesterone, 17α-Hydroxyprogesterone (Acetate and Caproate), Levonorgestrel, Lynestrenol, Medrogestone, Medroxyprogesterone (and Acetate), Megestrol Acetate, Melengestrol, Norethindrone (Acetate and Enanthate), Norethisterone, Norethynodrel, Norgesterone, Norgestimate, Norgestrel, Norgestrienone, 19-Norprogesterone, Norvinisterone, Pentagestrone, Progesterone, Promegestone, Quingestrone and Trengestone; and (c) androgenically effective hormones such as Aldosterone, Androsterone, Boldenone, Cloxotestosterone, Dehydroepiandrosterone, Fluoxymesterone, Mestanolone, Mesterolone, Methandrostenolone, Methyltestosterone, 17α-Methyltesteosterone, 17α-Methyltestosterone 3-Cyclopentyl Enol Ether, Norethandrolone, Normethandrone, Oxandrolone, Oxymesterone, Oxymetholone, Prasterone, Stanlolone, Stanozolol, Testosterone (Acetate, Enanthate, Isobutyrate, Propionate and Undecanoate), Testosterone 17-Chloral Hemiacetal, Testosterone 17β-Cypionate and Tiomesterone.

(E) anabolic steroids such as Androisoxazole, Androstenediol, Bolandiol, Bolasterone, Clostebol, Ethylestrenol. Formyldienolone, 4-Hydroxy-1 g-nortestosterone, Methandriol, Methenolone, Methyltrienolone, Nandrolone, Nandrolone Decanoate, Nandrolone p-Hexyloxyphenylpropionate, Nandrolone Phenpropionate, Norbolethone, Oxymesterone, Pizotyline, Quinbolone, Stenbolone and Trenbolone;

(F) anorexics such as Aminorex, Amphecloral, Amphetamine, Benzaphetamine, Chlorphentermine, Clobenzorex, Cloforex, Clortermine, Cyclexedrine, Destroamphetamine Sulfate, Diethylpropion, Diphemethoxidine, N-Ethylamphetamine, Fenbutrazate, Fenfluramine, Fenproporex, Furfurylmethylamphetamine, Levophacetoperate, Mazindol, Mefenorex, Metamfeproamone, Methamphetamine, Norpseudoephedrine, Phendimetrazine, Phendimetrazine Tartrate, Phenmetrazine, Phentermine, Phenylpropanola mine Hydrochloride and Picilorex;

(G) anticonvulsants such as Acetylpheneturide, Albutoin, Aloxidone, Aminoglutethimide, 4-Amino-3-hydroxybutyric Acid, Atrolactamide, Beclamide, Buramate, Calcium Bromide, Carbamazepine, Cinromide, Clomethiazole, Clonazepam, Decimemide, Diethadione, Dimethadione, Doxenitoin, Eterobarb, Ethadione, Ethosuximide, Ethotoin, Fluoresone, Garbapentin, 5-Hydroxytryptophan, Lamotrigine, Lomactil, Magnesium Bromide, Magnesium Sulfate, Mephenyloin, Mephobarbital, Metharbital, Methetoin, Methsuximide, 5-Methyl-5-(3-phenanthryl)hydantoin, 3-Methyl-5-phenylhydantoin Narcobarbital, Nimetazepam, Nitrazepam, Paramethadione, Phenacemide, Phenetharbital, Pheneturide, Phenobarbital, Phenobarbital Sodium, Phensuximide, Phenylmethylbarbituric Acid, Phenyloin, Phethenylate Sodium, Potassium. Bromide, Pregabatin, Primidone, Progabide, Sodium Bromide, Sodium Valproate, Solanum, Strontium Bromide, Suclofenide, Sulthiame, Tetrantoin, Tiagabine, Trimethadione, Valproic Acid, Valpromide, Vigabatrin and Zonisamide; and

(H) others including cocaine, coca derivatives, lysergic acid and lysergic acid amide.

The compositions and methods disclosed herein are not limited to therapeutic agents that are subject to abuse or that are precursors to abused substances and can include any type of therapeutic agent. Further types of therapeutic agents that can be used in the methods and compositions disclosed herein include, but are not limited to, α-adrenergic agonists, β-adrenergic agonists, α-adrenergic blockers, β-adrenergic blockers, alcohol deterrents, aldose reductase inhibitors, non-narcotic analgesics, anesthetics, anthelmintics, antiacne drugs, antiallergenics, antiamebics, antiandrogens, antianginals, antiarrhythmics, anticoagulants, anti-erectile dysfunction agents, anti-infectives, antioxidants, antiarteriosclerotics, antiarthritic/antirheumatics, antibacterial (antibiotic) drugs, antibacterial drugs (synthetic), anticholinergics, anticonvulsants, antidepressants, antidiabetics, antidiarrheal drugs, antidiuretics, antiestrogens, antifungal drugs (antibiotics), antifungal drugs (synthetic), antiglaucoma drugs, antigonadotropins, antigout drugs, antihistamines, antihyperlipoproteinemics, antihypertensive drugs, antihyperthyroids, antihypotensive drugs, antihypothyroid drugs, anti-Inflammatory (non-steroidal) drugs, antimalarial drugs, antimigraine drugs, antinauseant drugs, antineoplastic drugs, antineoplastic (hormonal) drugs, antineoplastic adjuncts, antiparkinsonian drugs, antipheochromocytoma drugs, antipneumocystis drugs, antiprostatic hypertrophy drugs, antiprotozoal drugs, antipuritics, antipsoriatic drugs, antipsychotic drugs, antipyretics, antirickettsial drugs, antiseborrheic drugs, antiseptics, antispasmodic drugs, antithrombotic drugs, antitussive drugs, antiulcerative drugs, antiurolithic drugs, antivenin drugs, antiviral drugs, anxiolytic drugs, benzodiazepine antagonists, bronchodilators, calcium channel blockers, calcium regulators, cardiotonics, chelating agents, cholecystokinin antagonists, cholelitholytic agents, choleretics, cholinergic agents, cholinesterase inhibitors, cholinesterase reactivators, central nervous system stimulants and agents, decongestants, dental agents, depigmentors, diuretics, dopamine receptor agonists, ectoparasiticides, enzymes, enzyme inducers (hepatic), estrogens (non-steroidal), gastric secretion inhibitors, glucocorticoids, gonad-stimulating principles, gonadotropic hormones, growth hormone inhibitors, growth hormone releasing factors, growth stimulants, hemolytic agents, heparin antagonists, hepatoprotectants, immunomodulators, immunosuppressants, ion exchange resins, lactation stimulating hormone, LH-RH agonists, lipotropic agents, lupus erythematosus suppressants, mineralcorticoids, miotic drugs, monoamine oxidase inhibitors, mucolytic agents, muscle relaxants (skeletal), narcotic antagonists, neuroprotective agents, nootropic agents, ophthalmic agents, ovarian hormone, oxytocic drugs, pepsin inhibitors, peristaltic stimulants, prolactin inhibitors, prostaglandins and prostaglandin analogs, protease inhibitors, respiratory stimulants, sclerosing agents, thrombolytic agents, thyrotropic hormones, uricosurics, vasodilators (cerebral), vasodilators (coronary), vasodilators (peripheral), chemotherapeutic agents, retinoids, antibiotics, desensitizing agents, vaccines, antiproliferatives, antiphotoaging agents, melanotropic peptides, radiation absorbers, parasympatholytics, sympatholytics, androgenic steroids, progestational agents, humoral agents, cardioactive agents, nutritional agents, and natural and synthetic bioactive peptides and proteins.

The amount of therapeutic agent in each oral dosage form will be determined based on the expected amount of therapeutic agent to be released and the release characteristics of the matrix. For example, for the opioid therapeutic hydromorphone hydrochloride, each oral dosage form may include at least 5 mg, at least 10 mg, at least 15 mg, or at least 20 mg. For hydromorphone, the oral dosage form may include less than about 40 mg of hydromorphone and/or pharmaceutically acceptable salts of hydromorphone.

Plasticizers

In preferred embodiments, a plasticizer is also included in the oral dosage form. Plasticizers interact with the hydrophobic matrix material resulting in a lower viscosity of the mixture during extrusion or molding. The result is that extrusion or injection molding of the oral dosage form can occur at lower temperatures, thereby reducing the possibility of thermally degrading the therapeutic agent. The most suitable plasticizers are those that lower the glass transition temperature (Tg) of the hydrophobic matrix material. Plasticizers suitable for use with the compositions and methods disclosed herein include, but are not limited to, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and allyl glycolate.

Excipients

In addition to a hydrophobic matrix material and a therapeutic agent, compositions may also include one or more functional excipients such as lubricants, thermal lubricants, antioxidants, buffering agents, alkalinizing agents, disintegrants, binders, diluents, sweeteners, chelating agents, colorants, flavorants, surfactants, solubilizers, wetting agents, stabilizers, hydrophilic polymers, hydrophobic polymers, waxes, lipophilic materials, absorption enhancers, preservatives, absorbents, cross-linking agents, bioadhesive polymers, retardants, pore formers, osmotic agents and fragrance.

Lubricants or thermal lubricants useful as an excipient include, but are not limited to fatty esters, glyceryl monooleate, glyceryl monostearate, wax, carnauba wax, beeswax, vitamin E succinate, and a combination thereof.

As used herein, the term “antioxidant” is intended to mean an agent that inhibits oxidation and thus is used to prevent the deterioration of preparations by oxidation due to the presence of oxygen free radicals or free metals in the composition. Such compounds include, by way of example and without limitation, ascorbic acid (Vitamin C), ascorbyl palmitate, butylated hydroxyanisole (BHA), butylated hydroxytoluene (BHT), hypophosphorous acid, monothioglycerol, sodium ascorbate, sodium formaldehyde sulfoxylate, sodium metabisulfite, sodium bisulfite, vitamin E and its derivatives, propyl gallate and others known to those of ordinary skill in the art.

Binders are ingredients added to mixtures to provide adhesive qualities during and after formation of an oral dosage. Examples of binders include, but are not limited to: waxes such as beeswax; carnauba wax; microcrystalline wax and paraffin wax; cetyl palmitate; glycerol behenate; glyceryl palmitostearate; glyceryl stearate; hydrogenated castor oil; stearic acid; stearic alcohol; stearate 6000 WL1644; gelucire 50/13; polyethylene glycols (PEG) such as PEG 2000, PEG 3000, PEG 6000, PEG 8000, PEG 10000, PEG 20000; polyethylene oxide; polypropylene oxide; polyvinylpyrrolidone; polyvinylpyrrolidone-co-vinylacetate; acrylate-methacrylate copolymers; polyethylene; polycaprolactone; alkylcelluloses such as methylcellulose; hydroxyalkylcelluloses such as hydroxymethylcellulose, hydroxyethylcellulose, hydroxypropylcellulose, and hydroxybutylcellulose; hydroxyalkyl alkylcelluloses such as hydroxyethyl methylcellulose and hydroxypropyl methylcellulose; starches, pectins; polylactic acid (PLA); polyglycolic acid (PLGA), polyesters (e.g., shellac); and polysaccharides such as cellulose, tragacanth, gum arabic, guar gum, and xanthan gum.

A buffering agent is used to resist change in pH upon dilution or addition of acid or alkali. Such compounds include, by way of example and without limitation, potassium metaphosphate, potassium phosphate, monobasic sodium acetate and sodium citrate anhydrous and dihydrate, salts of inorganic or organic acids, salts of inorganic or organic bases, and others known to those of ordinary skill in the art.

As used herein, the term “alkalizing agent” is intended to mean a compound used to provide alkaline medium for product stability. Such compounds include, by way of example and without limitation, ammonium carbonate, diethanolamine, monoethanolamine, potassium hydroxide, sodium, borate, sodium carbonate, sodium bicarbonate, sodium hydroxide, triethanolamine and others known to those of ordinary skill in the art.

As used herein, the term “disintegrant” is intended to mean a compound used in solid dosage forms to promote the disruption of a solid mass (layer) into smaller particles that are more readily dispersed or dissolved. Exemplary disintegrants include, by way of example and without limitation, starches such as corn starch, potato starch, pre-gelatinized and modified starches thereof, sweeteners, clays, bentonite, microcrystalline cellulose (e.g., Avicel™), carboxymethylcellulose calcium, croscarmellose sodium, alginic acid, sodium alginate, cellulose polyacrilin potassium (e.g., Amberlite™), alginates, sodium starch glycolate, gums, agar, guar, locust bean, karaya, pectin, tragacanth, crospovidone and other materials known to one of ordinary skill in the art. A superdisintegrant is a rapidly acting disintegrant. Exemplary superdisintegrants include crospovidone and low substituted HPC.

Exemplary chelating agents include EDTA, polyamines, derivatives thereof, and others known to those of ordinary skill in the art.

As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other FD&C dyes and natural coloring agents such as grape skin extract, beet red powder, beta carotene, annato, carmine, turmeric, paprika, and other materials known to one of ordinary skill in the art. The amount of coloring agent used will vary as desired.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful, flavors include vanilla, citrus oil, including lemon, orange, grape, lime and grapefruit, and fruit essences, including apple, pear, peach, strawberry, raspberry, cherry, plum, pineapple, apricot and so forth. Flavors that have been found to be particularly useful include commercially available orange, grape, cherry and bubble gum flavors and mixtures thereof. The amount of flavoring may depend on a number of factors, including the organoleptic effect desired. Flavors will be present in any amount as desired by those of ordinary skill in the art. Particular flavors are the grape and cherry flavors and citrus flavors such as orange.

Surfactants include soaps, synthetic detergents, and wetting agents. Suitable surfactants include cationic surfactants, anionic surfactants, non-ionic surfactants, and amphoteric surfactants. Examples of surfactants include Polysorbate 80; sorbitan monooleate; sodium lauryl sulfate (sodium dodecylsulfate); soaps such as fatty acid alkali metal salts, ammonium salts, and triethanolamine salts; cationic detergents such as dimethyl dialkyl ammonium halides, alkyl pyridinium halides, and alkylamine acetates; anionic detergents such as alkyl, aryl and olefin sulfonates, alkyl, olefin, ether and monoglyceride sulfates, and sulfosuccinates; nonionic detergents such as fatty amine oxides, fatty acid alkanolamides, and poly(oxyethylene)-block-poly(oxypropylene)copolymers; and amphoteric detergents, for example, alkyl β-aminopropionates and 2-alkylimidazoline quaternary ammonium salts; wetting agents such as, glycerin, proteins, and peptides; water miscible solvents such as glycols; and mixtures thereof.

Solubilizers include cyclodextrins, povidone, combinations thereof, and others known to those of ordinary skill in the art.

Exemplary hydrophilic polymers which can be a primary or secondary polymeric carrier that can be included in the composition ludeude poly(vinyl alcohol) (PVA), polyethylene-polypropylene glycol (e.g. poloxamer), carbomer, polycarbophil, or chitosan. Hydrophilic polymers include, but are not limited to, one or more of, carboxymethylcellulose, hydroxypropyl cellulose, hydroxyethyl cellulose, methylcellulose, natural gums such as gum guar, gum acacia, gum tragacanth, or gum xanthan and povidone. “Hydrophilic polymers” also include polyethylene oxide, sodium carboxymethycellulose, hydroxyethyl methyl cellulose, hydroxymethyl cellulose, carboxypolyymethylene, polyethylene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), carrageenate alginates, carbomer, ammonium alginate, sodium alginate, or mixtures thereof.

Exemplary hydrophobic polymers include alkylcelluloses, ethyl cellulose, Eudragit RS, waxes, polyesters, combinations thereof, and others known to those of ordinary skill in the art.

Exemplary waxes include carnauba wax, beeswax, microcrystalline wax and others known to one of ordinary skill in the art.

Exemplary absorption enhancers include dimethyl sulfoxide, Vitamin E PGS, sodium cholate and others known to one of ordinary skill in the art.

Preservatives include compounds used to prevent the growth of microorganisms. Suitable preservatives include, by way of example and without limitation, benzalkonium chloride, benzethonium chloride, benzyl alcohol, cetylpyridinium chloride, chlorobutanol, phenol, phenylethyl alcohol, phenylmercuric nitrate and thimerosal and others known to those of ordinary skill in the art.

Examples of absorbents include sodium starch glycolate (Explotab™, Primojel™); croscarmellose sodium (Ac-Di-Sol®); polyvinylpyrrolidone VP) (e.g., Polyplasdone™ XL 10); veegum; clays; alginates; alginic acid; carboxymethylcellulose calcium; microcrystalline cellulose (e.g., Avicel™); polacrillin potassium (e.g., Amberlite™); sodium alginate; corn starch; potato starch; pregelatinized starch; modified starch; cellulosic agents; montmorrilonite clays (e.g., bentonite); gums; agar: locust bean gum; gum karaya; pecitin; tragacanth; and other absorbents known in to those of ordinary skill in the art.

In an embodiment, the oral dosage form may include one or more polycarboxylic acids. Polycarboxylic acids include organic compounds that have two or more carboxyl (—COOH) groups and from 2 to 9 carbon atoms in a chain or ring to which the carboxyl groups are attached. The carboxyl groups are not included when determining the number of carbon atoms in the chain or ring (e.g., 1,2,3 propane tricarboxylic acid would be considered to be a C₃ polycarboxylic acid containing three carboxyl groups and 1,2,3,4 butanetetracarboxylic acid would be considered to be a C₄ polycarboxylic acid containing four carboxyl groups). C₂-C₉ polycarboxylic acids include, but are not limited to aliphatic, aromatic, and alicyclic acids, either saturated or olefinically unsaturated, with at least two carboxyl groups per molecule. In some embodiments, aliphatic polycarboxylic acids may include a hydroxyl group attached to a carbon atom alpha to a carboxyl group (an α-hydroxy polycarboxylic acid). α-hydroxy polycarboxylic acids include citric acid (also known as 2-hydroxy-1,2,3 propane tricarboxylic acid) and tartaric acid.

Examples of specific polycarboxylic acids include, but are not limited to, oxalic acid, malonic acid, succinic acid, glutaric acid, adipic acid, maleic acid, fumaric acid, malic acid, pimelic acid, nonanedioic acid, dodecanedioic acid, octanedioic acid, phthalic acid, isophthalic acid, terephthalic acid, citraconic (methylmaleic acid), citric acid, tartaric acid, itaconic acid (methylenesuccinic acid), 1,2,3 propane tricarboxylic acid, transaconitic acid (trans-1-propene-1,2,3-tricarboxylic acid), 1,2,3,4-butanetetracarboxylic acid, all-cis-1,2,3,4-cyclopentanetetracarboxylic acid, mellitic acid (benzenehexacarboxylic acid), oxydisuccinic acid (2,2′-oxybis(butanedioic acid), α-bromoglutaric acid, 3,3-dimethylpentanedioic acid, and 2,4-dicholoropentanedioic acid.

Bioadhesive polymers include polyethylene oxide, KLUCEL (hydroxypropylcellulose), CARBOPOL, polycarbophil, GANTREZ, Poloxamer, and combinations thereof, and others known to one of ordinary skill in the art.

Retardants are agents that are insoluble or slightly soluble polymers with a Tg above 45° C., or above 50° C. before being plasticized by other agents in the formulation including other polymers and other excipients needed for processing. The excipients include waxes, acrylics, cellulosics, lipids, proteins, glycols, and the like.

Exemplary pore formers include water soluble polymers such as polyethylene glycol, propylene glycol, and povidone; binders such as lactose, calcium sulfate, calcium phosphate and the like; salts such as sodium chloride, magnesium chloride and the like, poloxamers and combinations thereof and other similar or equivalent materials which are widely known in the art. Examples of poloxamers include, but are not limited to: Pluronice F-68 (Poloxamer 188), Pluronic® F87 (Poloxamer 237), Pluronic® F108 (Poloxamer 338), Pluronic® F127 (Poloxamer 407, Lutrol F127) and the like. Pluronic® is a registered tradename for BASF Corporation for block copolymers of ethylene oxide and propylene oxide represented by the chemical structure HO(C₂H₄O)ₐ(C₃H₆O)_{b}(C₂H₄O)ₐH wherein for: (a) Pluronic® F-68, a is 80 and b is 27; (b) Pluronic® F87, a is 64 and b is 37; (c) Pluronic® F108, a is 141 and b is 44; and Pluronic® F127, a is 101 and b is 56. The average molecular weights of these block copolymers are 8,400, 7,700, 14,600 and 12,600 for Pluronic® F-68, Pluronic® F-87, Pluronic® F108 and Pluronic® F127, respectively.

Exemplary osmagents or osmotic agents include organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, combinations thereof and other similar or equivalent materials which are widely known in the art.

As used herein, the term “sweetening agent” is intended to mean a compound used to impart sweetness to a preparation. Such compounds include, by, way of example and without limitation, aspartame, dextrose, glycerin, mannitol, saccharin sodium, sorbitol, sucrose, fructose and other such materials known to those of ordinary skill in the art.

It should be understood that compounds used as excipients or that are used to modify the oral dosage form, may serve a variety of functions or purposes. Thus, whether a compound named herein is assigned to one or more classifications or functions, its purpose or function should not be considered as being limited to the named purpose or function.

Emetics and Nasal Irritants

In certain embodiments the oral dosage form also includes an emetic. While the use of emetics to deter abuse is not required for the oral dosage forms described herein, they can provide an additional deterrent to abuse when used in combination with the other components of the oral dosage forms. In principle, the amount of emetic supplied must be low enough to produce no ill effects on a subject or patient when the oral dosage form containing the emetic is used properly, that is, swallowed whole. However when the dosage form is crushed or dissolved, the result will be to release an amount of emetic that will produce vomiting when the crushed or dissolved oral dosage form is ingested. Suitable emetics include but are not limited to denatonium benzoate, syrup of ipecac, potassium tartrate, copper sulfate, zinc sulfate, cephaeline, methyl cephaeline, psychotrine, O-methylpsychotrine and emetamine and others known to one of ordinary skill in the art.

Similarly, in some embodiments, the oral dosage form can also include a nasal irritant. Similar to emetics, use of nasal irritants to deter abuse is not required for the oral dosage forms described herein. Furthermore, the type and amount of nasal irritant present in the oral dosage form must be such that substantially no ill side effects on a subject or patient occur when the oral dosage form is ingested. However, when the dosage form is crushed and inhaled, the presence of the nasal irritant will result in sneezing or discomfort in the user that deters further abuse. Suitable nasal irritants for use include but are not limited to sodium lauryl sulfate, pepper, capsaicin, ethylene glycol, poloxamer, sorbitan monoesters and glyceryl monooleates and others known to one of ordinary skill in the art.

Methods of Formulation

Further provided are methods of formulating oral dosage forms. Oral dosage forms that deter abuse may be formulated by:

a. mixing one or more hydrophobic matrix materials and a therapeutic agent, wherein the hydrophobic matrix materials includes 20 to 99.9% of the mixture by weight;

b. melting the mixture;

c. permitting the mixture to solidify as a solid mass or a substantially solid oral dosage form, wherein the mass or oral dosage form weighs at least 40 mg,

d. and optionally, shaping the mass into an oral dosage form.

For purposes of the present disclosure a mixture is “melted” by applying thermal or mechanical energy sufficient to render the mixture partially or substantially completely molten. For instance, in a mixture that includes a matrix material, “melting” the mixture may include substantially melting the matrix material without substantially melting one or more other materials present in the mixture (e.g., the therapeutic agent and one or more excipients). Generally, a mixture is sufficiently molten, for example, when it can be extruded as a continuous rod, or when it can be subjected to injection molding.

In preferred embodiments the hydrophobic matrix material is a water-insoluble polymer.

The mixture of the hydrophobic matrix material, therapeutic agent, optional plasticizer, optional functional excipients and optional emetic or nasal irritant can be accomplished by any suitable means. Well-known mixing means known to those skilled in the art include dry mixing, dry granulation, wet granulation, melt granualation, high shear mixing, and low shear mixing.

Granulation generally is the process wherein particles of powder are made to adhere to one another to form granules, typically in the size range of 0.2 to 4.0 mm. Granulation is desirable in pharmaceutical formulations because it produces relatively homogeneous mixing of different sized particles.

Dry granulation involves aggregating powders under high pressure. Wet granulation involves forming granules using a granulating fluid or wetting agent that is subsequently removed by drying. Melt granulation is a process in which powders are transformed into solid aggregates or agglomerates while being heated. It is similar to wet granulation except that a binder acts as a wetting agent only after it has melted. All of these and other methods of mixing pharmaceutical formulations are well-known in the art.

Subsequent or simultaneous with mixing, the mixture of hydrophobic matrix material, therapeutic agent, optional plasticizer, optional functional excipients and optional emetic or nasal irritant is melted to produce a mass sufficiently fluid to permit shaping of the mixture and/or to produce melding of the components of the mixture. The melted mixture is then permitted to solidify as a substantially solid oral dosage form. The mixture can optionally be shaped or cut into suitable sizes during the melting step or during the solidifying step. In one embodiment, oral dosage forms are single substantially solid masses of at least 40 mgs, at least 60 mgs, at least 80 mgs, at least 100 mgs, at least 150 mgs, at least 200 mgs, at least 250 mgs, at least 300 mgs, at least 400 mgs or at least 500 mgs. As used herein, a substantially solid oral dosage form is a dosage form that cannot be readily crushed or divided by hand into smaller parts and that preferably has a hardness of at least 20 kp, at least 25 kp, at least 30 kp, at least 35 kp, at least, 40 kp, at least 45 kp, or at least 50 kp.

In preferred embodiments, the mixture becomes a homogeneous mixture either prior to or during the melting step.

Methods of melting the mixture include, but are not limited to, hot-melt extrusion, injection molding and compression molding.

Hot-melt extrusion typically involves the use of an extruder device. Such devices are well-known in the art. Such systems include mechanisms for heating the mixture to an appropriate temperature and forcing the melted feed material under pressure through a die to produce a rod, sheet or other desired shape of constant cross-section. Subsequent to or simultaneous with being forced through the die the extrudate can be cut into smaller sizes appropriate for use as an oral dosage form. Any suitable cutting device known to those skilled in the art can be used, and the mixture can be cut into appropriate sizes either while still at least somewhat soft or after the extrudate has solidified. The extrudate may be cut, ground or otherwise shaped to a shape and size appropriate to the desired oral dosage form prior to solidification, or may be cut, ground or otherwise shaped after solidification. In some embodiments, an oral dosage form may be made as a non-compressed hot-melt extrudate. In other embodiments, an oral dosage form is not in the form of a compressed tablet.

Under certain conditions, extrusion of a composition may result in “die-swelling,” a phenomenon in which the extrudate swells diametrically after exiting the die. In certain embodiments, die-swelling can be desirable, producing an extrudate having greater porosity and thus accelerated release characteristics. In other embodiments, it can be desirable to avoid die swelling, thereby producing a more solid composition that has slower therapeutic release and/or is slower to dissolve in a solvent such as aqueous ethanol solutions and/or is harder.

Injection molding typically involves the use of an injection-molding device. Such devices are well-known in the art. Injection molding systems force a melted mixture into a mold of an appropriate size and shape. The mixture solidifies as least partially within the mold and then is released.

Compression molding typically involves the use of an compression-molding, device. Such devices are well-known in the art. Compression molding is a method in which the mixture is optionally preheated and then placed into a heated mold cavity. The mold is closed and pressure is applied. Heat and pressure are typically applied until the molding material is cured. The molded oral dosage form is then released from the mold.

The oral dosage forms may be of any size suitable for oral administration. In some embodiments, oral dosage forms are roughly cylindrical in shape. In a plane perpendicular to the long axis of the cylinder the roughly cylindrical preferred oral dosage form has a diameter of 5 mm or greater, 6 mm or greater, 7 mm or greater, 8 mm or greater, 9 mm or greater, or 10 mm or greater. Along the long axis of the cylinder the preferred oral dosage form has a length of 1, 2, 3, 4, 5, 6, 7, 8, 9, 10, 11, 12, 13, 14 or 15 mm or greater. Such dosage forms could be formed, for example, by extruding the oral dosage form through a die that is at least 0.5 mm in diameter, 0.6 mm in diameter, 0.7 mm, etc., in diameter and then cutting the extrudate to a length of 1, 2, 3, 4, 5 mm, etc., in length.

It has been found, for some embodiments, that the release characteristics of the therapeutic agent from the oral dosage form may be dependent on the ratio of the surface area of the oral dosage form to the volume of the oral dosage form. In some embodiments, the surface area/volume ratio of the oral dosage form should be held constant to allow constant swelling and release of the therapeutic agent as the size of the oral dosage form is altered. In some embodiments, it is preferred that the surface are/volume ratio of the oral dosage form be maintained between about 0.5 to about 10, or between about 1 to about 5.

An oral dosage form produced by a thermal process may exhibit low moisture content. Reduced moisture content of the oral dosage form may improve the stability of the oral dosage form, thus extending the shelf life of the oral dosage form. In one embodiment, the oral dosage form has a moisture content of less than 5%, less than 4%, less than 3%, less than 2%, or less than 1%.

The final step in the process of making oral dosage forms is permitting the oral dosage form as a substantially solid oral dosage form, wherein the oral dosage form weighs at least 40 mg. The oral dosage form may optionally be shaped either prior to solidification or after solidification of the dosage form. Solidification will generally occur either as a result of cooling of the melted mixture or as a result of curing of the mixture however any suitable method for producing a solid dosage form may be used.

In certain embodiments, prior to administration the substantially solid oral dosage form may be cut, ground or otherwise shaped into its final form, or may be allowed to remain in its final molded configuration. Optionally the substantially solid oral dosage form can farther include one or more coatings, including polymeric coatings and the like.

In preferred embodiments, the oral dosage form includes a therapeutic agent as a substantially uniform solution or dispersion within a matrix of hydrophobic polymer. However in alternative embodiments the distribution of therapeutic agent within the hydrophobic polymer can be substantially non-uniform. One method of producing a non-uniform distribution of therapeutic agent is through the use of one or more coatings of water-insoluble or water-soluble polymer. Another method is by providing two or more mixtures of polymer or polymer and therapeutic agent to different zones of a compression or injection mold. A further method is by providing the therapeutic agent in form of particulates embedded in a matrix of 20-100% water-insoluble polymer by weight. These methods are provided by way of example and are not exclusive. Other methods of producing a non-uniform distribution of therapeutic agent within the abuse-deterring oral dosage forms will be apparent to those skilled in the art.

Release Characteristics

Previous uses in the art of hot-melt extrudates and other polymeric solids containing agents have involved providing a unit dosage form including the solid in the form of particulates, pellets, granules, or the like. This is because the use of particulates substantially increases the surface area of the unit dosage form. It was widely believed that such increased surface area was required to achieve sufficient drug release upon ingestion to make the dosage form suitable for pharmaceutical use. Oral dosage forms consisting essentially of a substantially-solid mass were not disclosed as oral dosage forms because the surface area of such dosage forms was considered to be inadequate for sufficiently rapid release of the embedded therapeutic agent.

Surprisingly, compositions described herein are suitable for immediate release, controlled release and extended release applications, or combinations thereof, depending on the types of hydrophobic matrix materials, therapeutic agent, plasticizers and excipients used and their proportions. Methods for adjusting these characteristics will be apparent to those skilled in the art or can be determined without undue experimentation. For example, immediate release characteristics of the oral dosage forms may be enhanced by the inclusion of hydrophilic therapeutic agents, plasticizers and/or excipients to enhance the formation of pores in the oral dosage form, particularly those that begin forming when the oral dosage form is subjected to gastric conditions. Alternatively, immediate release characteristics may be suppressed, for example, by coating the oral dosage form with a suitable enteric coating that does not contain the therapeutic agent. By adjusting variables such as these, a range of release characteristics can be obtained from the oral dosage forms.

In one embodiment, the oral dosage form releases at least 80% of the therapeutic agent after 2 hours of stirring in a 0.1 N HCl solution and 16 hours stirring in a pH 6.8 phosphate buffer solution using a USP Type II paddle apparatus at 75 rpm and 37° C. The oral dosage form, in some embodiments, releases between about 10% and about 50% of the therapeutic agent after 2. hours of stirring in a 0.1 N HCl solution and 1 hour stirring in a pH 6.8° phosphate buffer solution using a USP Type II paddle apparatus at 75 rpm and 37° C. The oral dosage form, in some embodiments, releases between about 40% and about 70% of the therapeutic agent after 2 hours of stirring in a 0.1 N HCl solution and 10 hours stirring in a pH 6.8 phosphate buffer solution using a USP Type II paddle apparatus at 75 rpm and 37° C. The oral dosage form, in some embodiments, releases between about 70% and about 100% of the therapeutic agent after 2 hours of, stirring in a 0.1 N HCl solution and 16 hours stirring in a pH 6.8 phosphate buffer solution using a USP Type II paddle apparatus at 75 rpm and 37° C.

In some embodiments, it has been found that the release characteristics and the abuse deterrent properties of a monolithic oral dosage form may be accomplished without the use of digestible C₈-C₅₀ substituted and unsubstituted hydrocarbons. Thus oral dosage formulation may be used that are substantially free of digestible C₈-C₅₀ substituted and unsubstituted hydrocarbons such as C₈-C₅₀ fatty acids, C₈-C₅₀ fatty alcohols, glyceryl esters of C₈-C₅₀ fatty acids, mineral oils, vegetable oils and waxes.

In some embodiments, the oral dosage form may be disposed in a capsule. Examples of materials that may be used to encapsulate the oral dosage form include, but are not limited to, gelatin capsules, hydroxypropylmethyl cellulose (“HPMC”) capsules, or polysaccharide capsules (e.g., pullulan capsules). In other embodiments, the oral dosage forms may be coated. Examples of coating materials include gelatins, aesthetic polymers, proteins or polysaccharides (e.g., sucrose). For testing of a coated or capsulated oral dosage form, the coating or capsule may be removed (e.g., by dissolving in an acidic solution) prior to performing an release or abuse deterrent test.

In some embodiments of the oral dosage form, it will be desirable to formulate compositions that they have specific release characteristics for treatment of a human or animal. Formulations of the oral dosage form, by their nature, lend themselves to immediate and extended-release applications. Not to be limited by theory, it is believed that the release characteristics of the oral dosage forms are a function of the solubility of the drug and the matrix in the gastric and intestinal milieu. It is anticipated that in some embodiments, drug release in the gastric milieu will be limited to diffusion of drug particles on the surface of the matrix, and that drug release from the matrix in the intestinal milieu will occur slowly by erosion and diffusion. For example, the release characteristics can be adjusted by one of ordinary skill in the art by use of pore formers, hydrophilic polymers, osmotic agents, plasticizers and other functional excipients. The chemical and physical properties, including the release characteristics, of the dosage form can also be adjusted by the process, processing parameters (temperature, shear rate) and equipment design (melt pump or rotating screw). Methods of adapting the oral dosage form to different therapeutic agents and different release profiles are routine in the art and can be accomplished without undue experimentation.

Methods of Deterring Drug Abuse

In an embodiment, a method of preventing drug abuse includes:
a. identifying a therapeutic agent that is subject to abuse;
b. formulating an oral dosage form that has a hardness of at least about 20 kp or greater and which releases less than about 40% of the therapeutic agent after 3 hours of shaking on an orbital shaker at 240 cycles/min in an aqueous solution of 40% ethanol at room temperature; and
c. providing the oral dosage form to a patient.

In an embodiment, an oral dosage form is formulated to have a hardness of at least about 20 kp, at least about 25 kp, at least about 30 kp, at least about 35 kp, at least about 40 kp, at least about 45 kp, or at least about 50 kp. In an embodiment, an oral dosage form is formulated to have a release of less than about 40%, less than about 30%, less than about 20% or less than about 10% of the therapeutic agent after 3 hours of shaking on an orbital shaker at 240 cycles/min in an aqueous solution of 40% ethanol at room temperature.

The resulting oral dosage forms are highly resistant to crushing and to dissolution in an ethanol solution such as a typical alcoholic beverage. As a result an abuser is deterred from bypassing the extended-release characteristics of the formulation such that they receive a single concentrated dose of the therapeutic agent.

In further embodiments, methods of deterring abuse include:

a. mixing one or more hydrophobic matrix materials and a therapeutic agent that is subject to abuse, wherein the hydrophobic matrix materials includes 20 to 99.9% of the mixture by weight;

b. melting the mixture;

c. permitting the mixture to solidify as a substantially solid mass or as a substantially solid oral dosage form, wherein the mass or oral dosage form weighs at least 40 mg;

d. optionally, shaping the mass into a substantially solid oral dosage form;

e. and administering the oral dosage form to a patient.

In certain embodiments, oral dosage forms that are resistant to ethanol extraction or dose-dumping in ethanol are disclosed. The disclosed formulations are also resistant to opioid abuse by including a therapeutic amount of an opioid agent and an effective amount of an opiod antagonist. The opioid antagonist is sequestered from the opioid agent such that the antagonist has no significant effect on the activity of the opioid when the dosage form is taken orally as prescribed. Tampering with the dosage form, or crushing the dosage form however, releases the antagonist in an amount effect to reduce the abuse potential of the opioid agent.

An antagonist is a drug or medication that prevents molecules of other drugs/medications from binding to a receptor (e.g., an opioid receptor). Antagonists can also displace other opioids and can precipitate withdrawal, or block the effects of other opioids. Opioid antagonists suitable for the present formulations include any opioid antagonist known in the art, mixed agonist/antagonists and partial antagonists. Such agents include but are not limited to naloxone, cyclazocine, naltrexone, nalmephene, alvimopan, nalide, nalmexone, nalorphine, nalorphine dinicotinate, and levallorphan, or the pharmacologically effective esters or salts of any of the foregoing antagonists.

Further provided are methods of formulating the oral dosage forms. Oral dosage forms that deter abuse are formulated by: mixing one or more hydrophobic matrix materials, an opioid agent, and a coated opioid antagonist, wherein the hydrophobic matrix materials comprises 20 to 99.9% of the mixture by weight; melting the mixture; permitting the mixture to solidify as a solid mass or oral dosage form, wherein the mass or oral dosage form weighs at least 40 mg; optionally, shaping the mass into a monolithic oral dosage form; and, optionally, over-encapsulating or coating the mass or oral dosage form in a shell.

The coated particles or microparticles of opioid antagonist may be prepared by various methods known in the art, including but not limited to hot-melt extrusion, compression molding or injection molding as described previously herein for production of the monolithic dosage forms. Other types of coatings for the opioid antagonists can include coatings that are pH dependent or pH independent, such as coatings formed from acrylic polymers, cellulose derivate polymers, waxes, or curable polymers, for example. Any coatings known in the art can be used, so long as the opioid antagonist is not released simultaneously with the opioid agent when placed in simulated gastric juice, but is released when the dosage form is crushed.

pH dependent coatings can include coatings formed from any of shellac, cellulose acetate phthalate (CAP), polyvinyl acetate phthalate (PVAP), hydroxypropylmethylcellulose phthalate, and methacrylic acid ester copolymers, or zein, for example. Hydrophobic polymeric coatings include coatings formed from acrylic polymers, acrylic copolymers, methacrylic polymers or methacrylic copolymers, including but not limited to Eudragit® L100, Eudragit® L100-55, Eudragit® L 30 D-55, Eudragit® S100, Eudragit® 4135F, Eudragit® RS, acrylic acid and methacrylic acid copolymers, methyl methacrylate, methyl, methacrylate copolymers, ethoxyethyl methacrylates, cyanoethyl methacrylates, aminoalkyl methacrylate copolymers, polyacrylic acid, polymethacrylic acid, methacrylic acid alkylamine copolymers, polymethyl methacrylate, polymethacrylic acid anhydride, polymethacrylate, polyacrylamide, polymethacrylic acid anhydride and glycidyl methacrylate copolymers, an alkylcellulose such as ethylcellulose, methylcellulose, carboxymethyl cellulose, hydroxyalkylcellulose, hydroxypropyl methylcelluloses such as hydroxypropyl methylcellulose phthalate, and hydroxypropyl methylcellulose acetate succinate, cellulose acetate butyrate, cellulose acetate phthalate, and cellulose acetate trimaleate, polyvinyl acetate phthalate, polyester, waxes, shellac, zein, or the like. The coating of the opioid antagonist particles can also include hydrophilic materials such as a pharmaceutically-acceptable, water-soluble polymers such as polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, carboxymethyl cellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin or the like.

Oral dosage forms may be produced by mixing the hydrophobic matrix material, opioid agent, opioid antagonist, optional plasticizer, optional functional excipients and optional emetic or nasal irritant by any suitable means. Well-known mixing means known to those skilled in the art include dry mixing, dry granulation, wet granulation, melt granulation, high shear mixing, and low shear mixing.

Subsequent or simultaneous with mixing, the mixture of hydrophobic matrix material, opioid agent, opioid antagonist, optional plasticizer, optional functional excipients and optional emetic or nasal irritant is melted to produce a mass sufficiently fluid to permit shaping of the mixture and/or to produce melding of the components of the mixture. The melted mixture is then permitted to solidify as a solidified oral dosage form. The mixture can optionally be shaped or cut into suitable sizes during the melting step or during the solidifying step. Oral dosage forms may be a single solidified mass of at least 40 mgs, at least 60 mgs, at least 80 mgs, at least 100 mgs, at least 150 mgs, at least 200 mgs, at least 250 mgs, at least 300 mgs, at least 400 mgs or at least 500 mgs.

Methods of preventing drug abuse are disclosed that includes: formulating a monolithic oral dosage form comprising an opioid agent and an opioid antagonist, wherein the dosage form has a weight of at least 40 mg; and wherein the dosage form releases less than about 40% of the opioid agent after 3 hours of shaking on an orbital shaker in an aqueous solution of 40% ethanol at room temperature and further wherein the opioid antagonist is sequestered from the opioid agent such that the antagonist has no significant effect on the activity of the opioid when the dosage form is taken orally as prescribed, but wherein the antagonist is released in an amount effective to reduce the abuse potential of the opioid agent contained in the dosage form when the dosage form is crushed; and optionally providing the oral dosage form to a patient.

In further embodiments, methods of deterring abuse include: mixing one or more hydrophobic matrix materials, an opioid agent and a coated opioid antagonist, wherein the hydrophobic matrix materials comprises 20 to 99.9% of the mixture by weight; melting the mixture; permitting the mixture to solidify as a solidified mass or as a solidified oral dosage form, wherein the mass or oral dosage form weighs at least 40 mg; optionally, shaping the mass into a monolithic oral dosage form; and optionally administering or providing the oral dosage form to a patient.

Further embodiments relate to methods of treating a number of conditions and diseases, particularly the treatment of pain. The methods include preparing oral dosage forms comprising at least 20% by weight of one or more hydrophobic materials or water-insoluble polymers and one or more opioid agents, and one or more coated opioid antagonists. Certain methods further include providing said oral dosage forms to a patient in need of treatment for a disease or a condition.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes' can be made in the specific embodiments which are disclosed and still obtain a like or similar result without departing from the spirit and scope of the invention.

### Example 1

Eudragit L100 was mixed with Triethyl Citrate, Cellulose Acetate Butyrate and Metronidazole in the amounts listed in Table I. While metronidazole is not ordinary considered a drug that is subject to abuse, it was used as a model in the present example because it is highly soluble in water and aqueous ethanol solutions.

The mixture was dry blended and the resultant blend was hot melt extruded into rods using a Davis Standard 1.25 inch single screw extruder operating at 90-150° C. equipped with a ⅜″ die which were subsequently cut into 200 mg tablets.

TABLE I

| Ingredient | % w/w |
|---|---|
| Eudragit L100 | 60 |
| Triethyl Citrate | 15 |
| Cellulose Acetate Butyrate | 20 |
| Metronidazole | 5 |

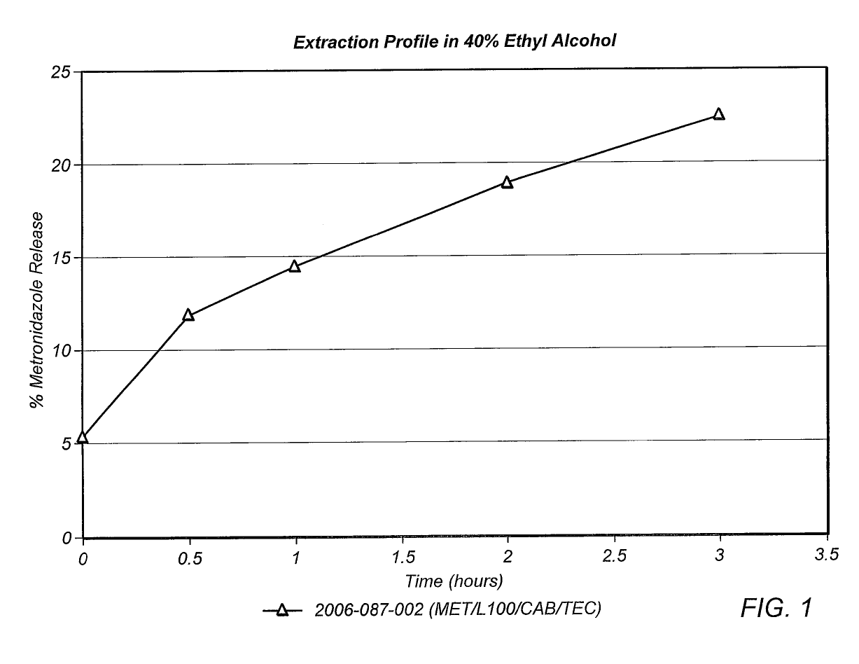

The tablets were placed into 4 ounce containers with 36 mL 0.1N HCl and shaken using an orbital shaker for 5 minutes at room temperature. Twenty four mL of Ethanol (100%) was added to the HCl solution to adjust the final alcohol concentration to 40% and shaking was continued for 3 hours. Samples of the ethanol solution were withdrawn and tested for metronidazole content throughout the run. FIG. 1 shows that at the conclusion of the test, less than 25% of the therapeutic agent was released from the dosage. It is to be expected that formulations containing therapeutic agents with aqueous or ethanol solubility less than metronidazole would release even less of the therapeutic agent under the same conditions.

The hardness of the resultant tablets was measured and was determined to be greater than 40 kp.

### Example 2

Water-insoluble polymer (ethyl cellulose) was used to prepare an oral dosage form also including water-soluble polymers (cellulose, carbomer and polyethylene oxide).

TABLE II

| Ingredient | % w/w |
|---|---|
| Oxycodone | 5 |
| Hydroxypropyl Cellulose (Klucel HF) | 51 |
| Dibutyl Sebacate | 9 |
| Vitamin E Oil | 1 |
| Ethyl Cellulose | 25 |
| Polyethylene Oxide | 4 |
| Carbomer | 5 |

The ingredients of Table II were blended and introduced to an extruder. Dibutyl sebacate is a plasticizer. Rods were extruded with a screw speed of 25 rpm and the extruder zones were heated to the temperatures listed in Table III. The resultant rods were cut into 400 mg tablets.

TABLE III

| Extruder Zones | Temperature |
|---|---|
| Zone 1 | 110° C. |
| Zone 2 | 110° C. |
| Zone 3 | 115° C. |
| Die | 115° C. |

After solidification the tablets were analyzed for their alcohol extractability in 40% ethanol with an orbital shaker for 3 hours at 240 cycles/min. The tablets were placed into 4 ounce containers with 36 mL 0.1N HCl and shaken using an orbital shaker for 5 minutes at room temperature. Twenty four mL of Ethanol (100%) was added to the HCl solution to adjust the final alcohol concentration to 40% and shaking was continued for 3 hours. Less than 40% of the oxycodone was released in 3 hours.

The hardness of the resultant tablets was measured and was determined to be greater than 30 kp.

### Example 3

Ethocel STD 100 (Dow Chemical) was mixed with Diethyl Phthalate, Hydroxypropyl Cellulose-HF (Aqualon), Lutrol F127 Micro (BASF), Citric Acid, Silicon Dioxide and Hydromorphone HCl in the amounts listed in Table IV.

TABLE IV

| Ingredient | % w/w |
|---|---|
| Ethocel STD 100 | 44.5 |
| Diethyl Phthalate | 20 |
| Hydroxypropyl Cellulose-HF | 10 |
| Lutrol F127 Micro | 5 |
| Citric Acid | 10 |
| Hydromorphone HCl | 10 |
| Silicon Dioxide | 0.5 |

The ingredients of Table IV were blended and introduced to an extruder. Rods, were extruded and cut into 100 mg and 300 mg tablets.

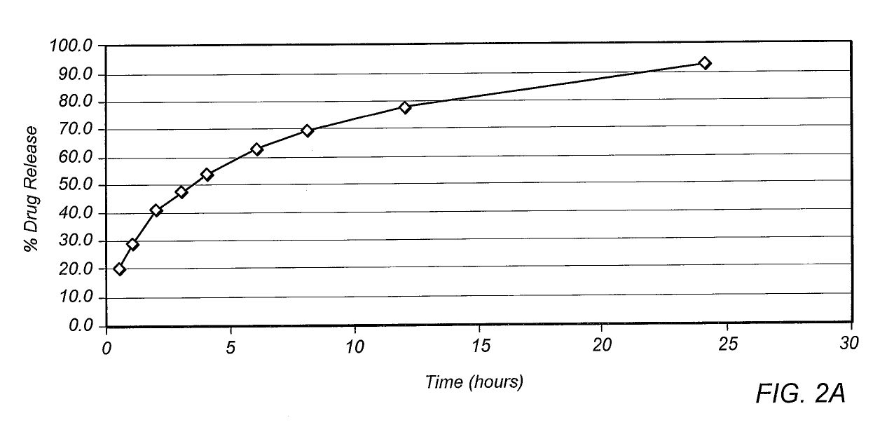

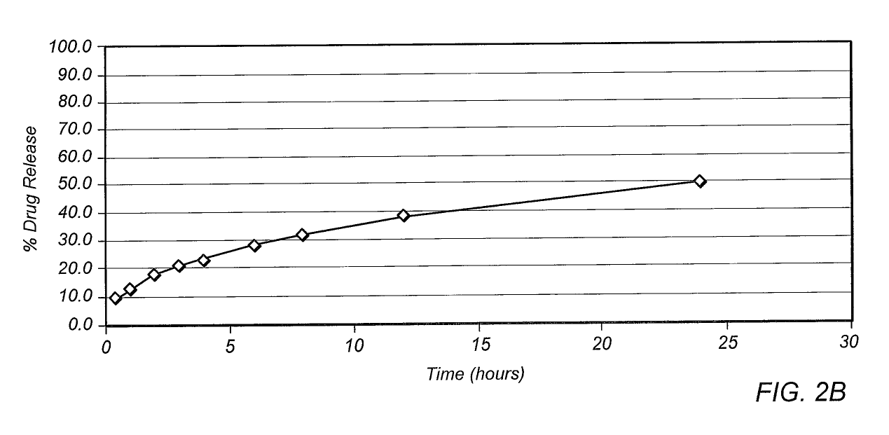

The rate at which the tablets dissolve, and thus release the hydromorphone HCl, was determined. The 100 mg and 300 mg tablets were placed in 750 mL of 0.1 N HCl and stirred for 2 hours. After this time, the pH was adjusted to 6.8 with phosphate buffer and stirred for 22 hours using a USP Type II paddle apparatus at 75 rpm and 37° C. The drug release profile for the 100 mg tablets is shown in FIG. 2A and the drug release profile for the 300 mg tablets is shown in FIG. 2B.

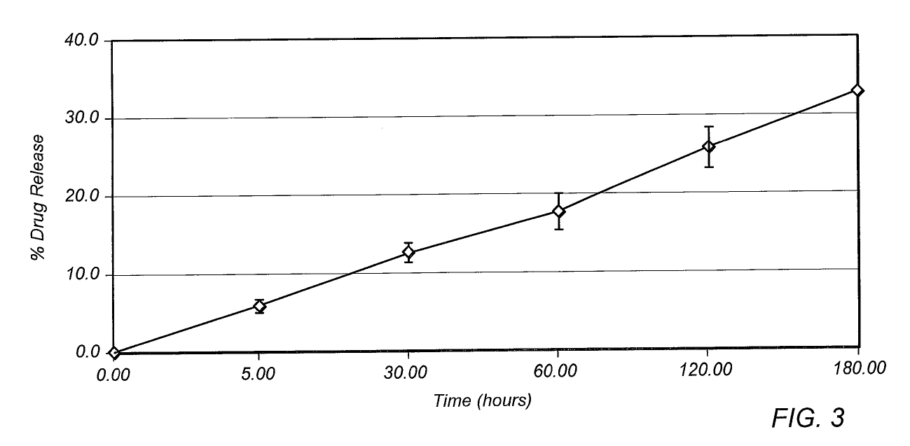

Abuse resistance (i.e., small volume alcohol extraction) of the tablets was investigated. The 100 mg tablets were placed into 4 ounce containers with 36 mL 0.1N HCl and shaken using an orbital shaker for 5 minutes at room temperature. Twenty four mL of Ethanol (100%) was added to the HCl solution to adjust the final alcohol concentration to 40% and shaking was continued for 3 hours at a rate of 240 cycles/min. The concentration of hydromorphone extracted into the aqueous ethanol solution at various time intervals was determined. The extraction results for the 100 mg tablets are shown in FIG. 3.

The hardness of the resultant tablets was measured and was determined to be greater than 50 kp.

### Example 4

Ethocel STD 100 (Dow Chemical) was mixed with Dibutyl Sebacate, Hydroxy Cellulose-HF (Aqualon), Lutrol F127 Micro (BASF), Citric Acid, Silicon Dioxide and Hydromorphone HCl in the amounts listed in Table V.

TABLE V

| Ingredient | % w/w |
|---|---|
| Ethocel STD 100 | 44.5 |
| Dibutyl sebacate | 20 |
| Hydroxypropyl Cellulose-HF | 15 |
| Lutrol F127 Micro | 5 |
| Citric Acid | 10 |
| Hydromorphone HCl | 5 |
| Silicon Dioxide | 0.5 |

The ingredients of Table V were blended and introduced to an extruder. Rods were extruded and cut into 200 mg tablets.

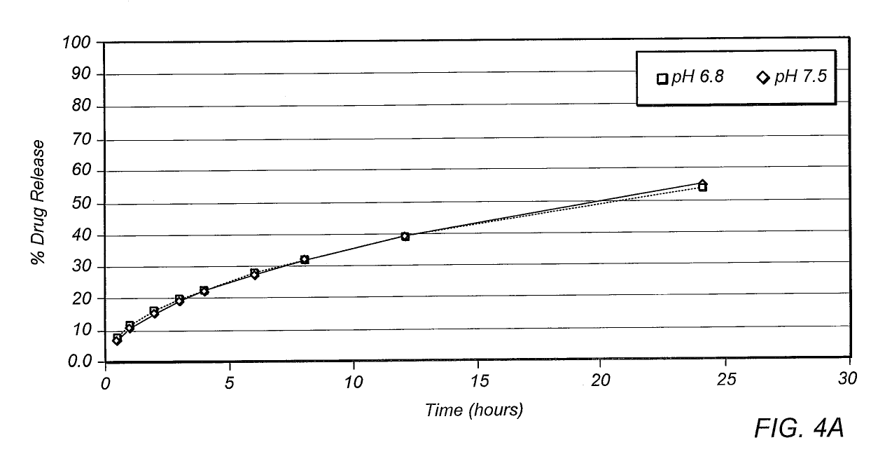

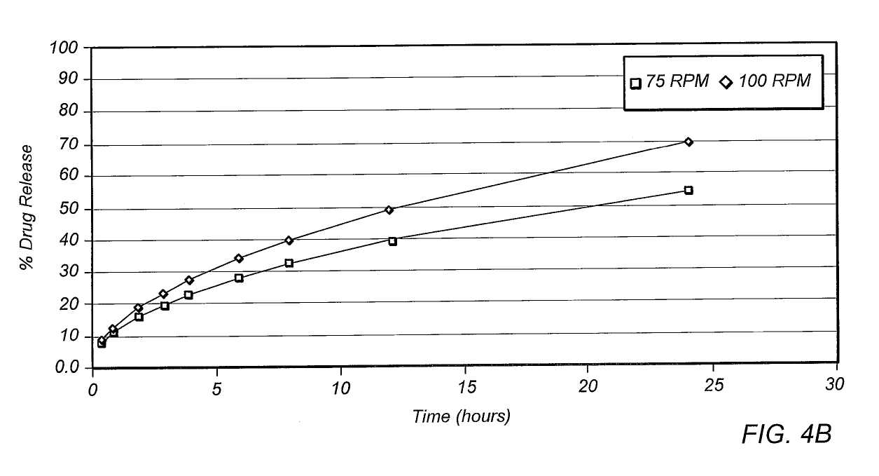

The rate at which the tablets dissolve, and thus release the hydromorphone HCl, was determined for three tablets. Each 200 mg tablets were placed in 750 mL of 0.1 N HCl and stirred for 2 hours. After this time, the pH of the mixture containing the first tablet was adjusted to pH 6.8 with phosphate buffer and stirred for 22 hours using a USP Type II paddle apparatus at 75 rpm and 37° C. The pH of the mixture containing the second tablet was adjusted to pH 7.5 with phosphate buffer and stirred for 22 hours using a USP Type II paddle apparatus at 75 rpm and 37° C. The pH of the mixture containing the third tablet was adjusted to pH 7.5 with phosphate buffer and stirred for 22 hours using a USP Type II paddle apparatus at 100 rpm and 37° C. The drug release profiles for the second and third tablets are shown in FIG. 4A. The drug release profiles for the first and second tablets are shown in FIG. 4B.

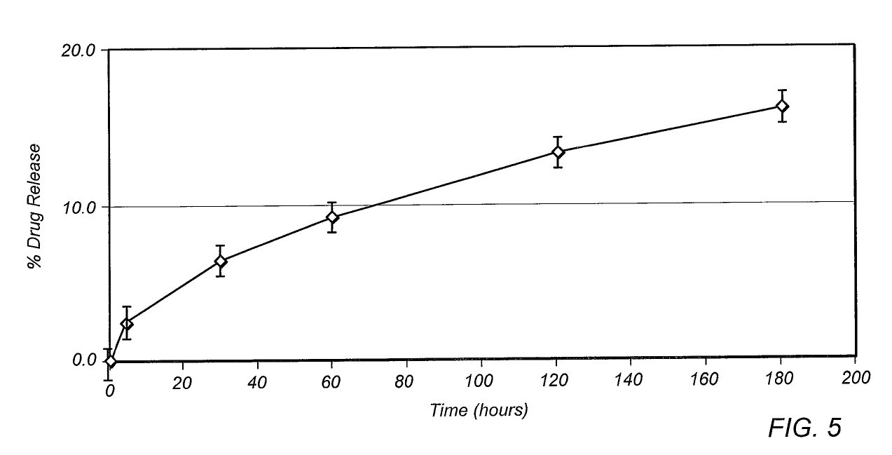

Abuse resistance (i.e., small volume alcohol extraction) of the tablets was investigated. The 100 mg tablets were placed into 4 ounce containers with 36 mL 0.1N HCl and shaken using an orbital shaker for 5 minutes at room temperature. Twenty four mL of Ethanol (100%) was added to the HCl solution to adjust the final alcohol concentration to 40% and shaking was continued for 3 hours at a rate of 240 cycles/min. The concentration of hydromorphone extracted into the aqueous ethanol solution at various time intervals was determined. The extraction results for the 200 mg tablets are shown in FIG. 5. The hardness of the resultant tablets was measured and was determined to be greater than 50 kp.

### Example 5

A composition was prepared with the compounds listed in Table VI.

TABLE VI

| Ingredient | % w/w |
|---|---|
| Ethocel STD 100 | 29 |
| Dibutyl sebacate | 10 |
| Castor Oil | 2.5 |
| Hydroxypropyl Cellulose-EF | 3.5 |
| Hydroxypropyl Cellulose-HF | 15 |
| Eudragit L100-55 | 15 |
| Sodium Dodecyl Sulfate | 1 |
| Citric Acid | 10 |
| Hydromorphone HCl | 10 |
| Talc | 3.5 |
| Silicon Dioxide | 0.5 |

The ingredients of Table VI were blended and introduced to an extruder. Rods were extruded and cut into 100 mg tablets.

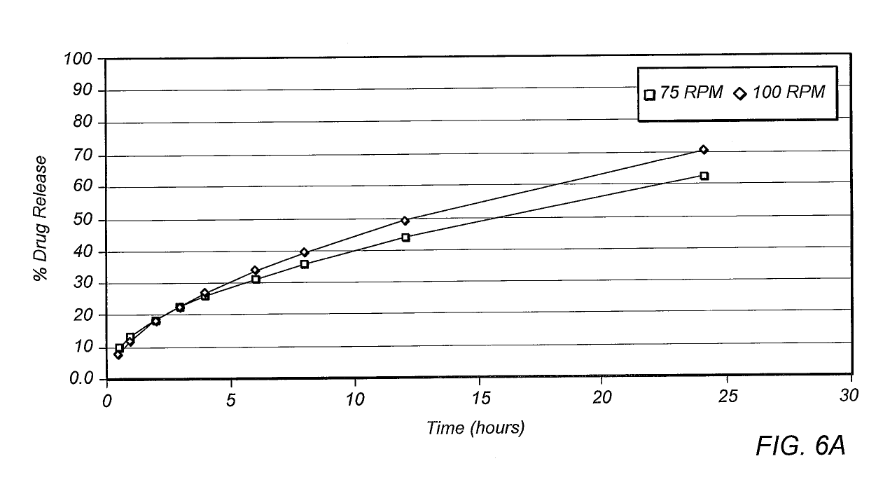

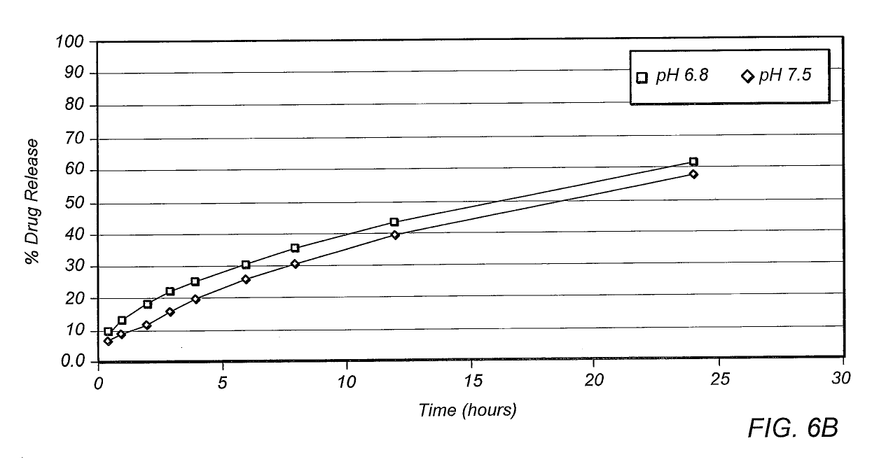

The rate at which the tablets dissolve, and thus release the hydromorphone HCl, was determined for three tablets. Each of the 100 mg tablets were placed in 750 mL of 0.1 N HCl and stirred for 2 hours. The pH of the mixture containing the first tablet was adjusted to pH 6.8 with phosphate buffer and stirred for 22 hours using a USP Type II paddle apparatus at 75 rpm and 37° C. The pH of the mixture containing the second tablet was adjusted to pH 7.5 with phosphate buffer and stirred for 22 hours using a USP Type II paddle apparatus at 75 rpm and 37° C. The pH of the mixture containing the third tablet was adjusted to pH 7.5 with phosphate buffer and stirred for 22 hours using a USP Type II paddle apparatus at 100 rpm and 37° C. The drug release profiles for the second and third tablets are shown in FIG. 6A. The drug release profiles for the first and second tablets are shown in FIG. 6B.

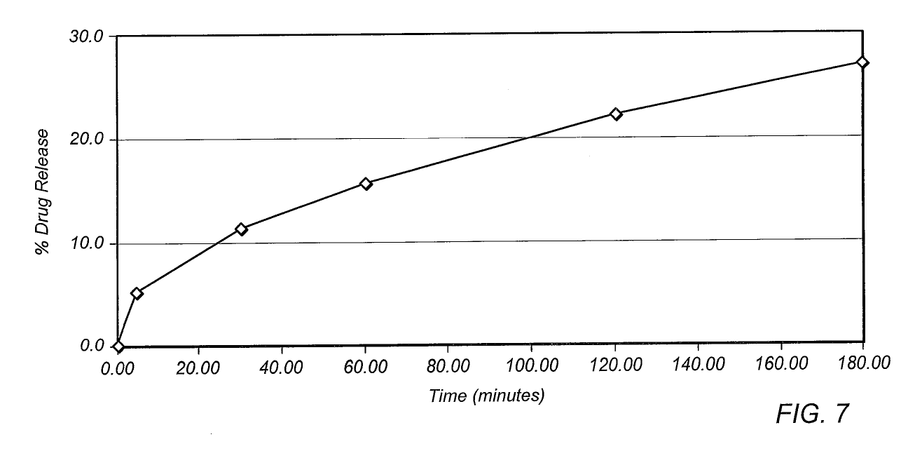

Abuse resistance (i.e., small volume alcohol extraction) of the tablets was investigated. The 100 mg tablets were placed into 4 ounce containers with 36 mL 0.1N HCl and shaken using an orbital shaker for 5 minutes at room temperature. Twenty four mL of Ethanol (100%) was added to the HCl solution to adjust the final alcohol concentration to 40% and shaking was continued for 3 hours at a rate of 240 cycles/min. The concentration of hydromorphone extracted into the aqueous ethanol solution at various time intervals was determined. The extraction results for the 100 mg tablets are shown in FIG. 7.

The hardness of the resultant tablets was measured and was determined to be greater than 50 kp.

### Example 6

Ethocel STD 100 (Dow Chemical) was mixed with Dibutyl Sebacate, Hydroxy Cellulose-HF (Aqualon), Poloxamer 407, Citric Acid, Silicon Dioxide and Hydromorphone HCl in the amounts listed in Table VII.

TABLE VII

| Ingredient | % w/w |
|---|---|
| Ethocel STD 100 | 39.5 |
| Dibutyl sebacate | 20 |
| Hydroxypropyl Cellulose-HF | 15 |
| Poloxamer 407 | 5 |
| Citric Acid | 10 |
| Hydromorphone HCl | 10 |
| Silicon Dioxide | 0.5 |

The ingredients of Table VII were blended and introduced to an extruder. Rods were extruded and cut into 100 mg tablets.

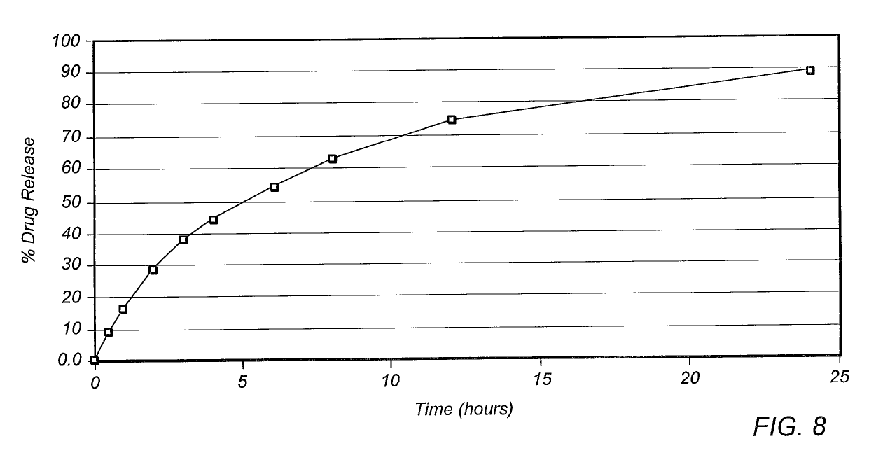

The rate at which the tablets dissolve, and thus release the hydromorphone HCl, was determined. A 100 mg tablet was placed in 750 mL of 0.1 N HCl and stirred for 2 hours. After this time, the pH of the mixture was adjusted to pH 6.8 with phosphate buffer and stirred for 22 hours using a USP Type II paddle apparatus at 75 rpm and 37° C. The drug release profile for the 100 mg tablet is shown in FIG. 8.

The hardness of the resultant tablets was measured and was determined to be greater than 50 kp.

### Example 7

Ethocel STD 100 (Dow Chemical) was mixed with Dibutyl Sebacate, Hydroxy Cellulose-HF (Aqualon), Lutrol F127 Micro (BASF), Citric Acid, Silicon Dioxide and Hydromorphone HCl in the amounts listed in Table VIII.

TABLE VIII

| Ingredient | % w/w |
|---|---|
| Ethocel STD 100 | 39.5 |
| Dibutyl sebacate | 20 |
| Hydroxypropyl Cellulose-HF | 15 |
| Lutrol F127 Micro | 5 |
| Citric Acid | 10 |
| Hydromorphone HCl | 10 |
| Silicon Dioxide | 0.5 |

The ingredients of Table VIII were blended and introduced to an extruder. Rods were extruded and cut into 200 mg tablets having a diameter of 5 mm or 6.5 mm.

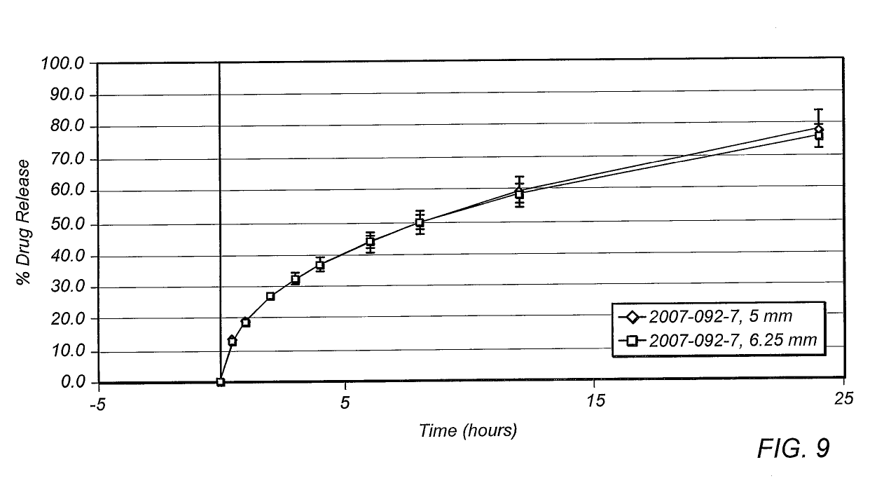

The rate at which the tablets dissolve, and thus release the hydromorphone HCl, was determined for 5 mm diameter and 6.5 mm diameter tablets. Each tablet was placed in 750 mL of 0.1 N HCl and stirred for 2 hours. After this time, the pH of each of the mixtures containing the tablets was adjusted to pH 6.8 with phosphate buffer and stirred for 22 hours using a USP Type II paddle apparatus at 75 rpm and 37° C. The drug release profiles for the 5.5 mm and the 6.5 mm tablets are shown in FIG. 9.

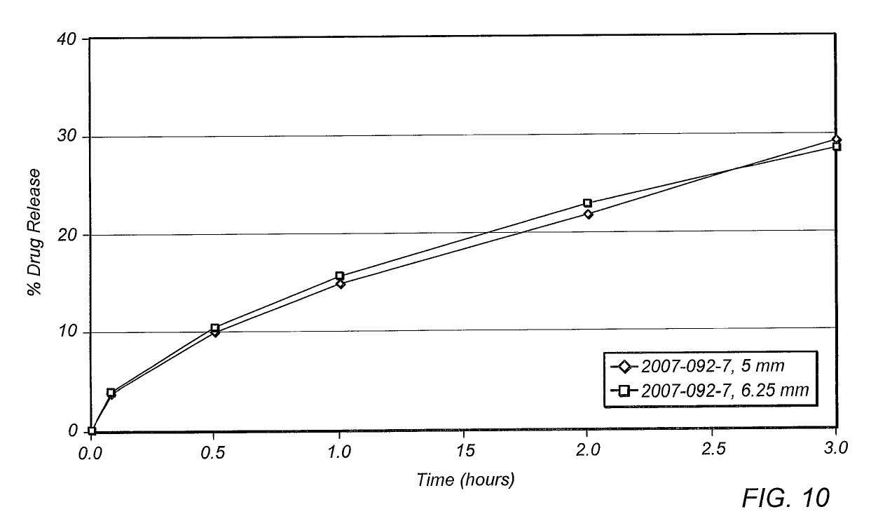

Abuse resistance (i.e., small volume alcohol extraction) of the tablets was investigated. The 5 mm and 6.5 mm, 200 mg tablets were placed into separate 4 ounce containers with 36 mL 0.1N HCl and shaken using an orbital shaker for 5 minutes at room temperature. Twenty four mL of Ethanol (100%) was added to the HCl solution to adjust the final alcohol concentration to 40% and shaking was continued for 3 hours at a rate of 240 cycles/min. The concentration of hydromorphone extracted into the aqueous ethanol solution at various time intervals was determined. The extraction results for the 5 mm and 6.5 mm tablets are shown in FIG. 10.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An oral dosage form comprising:
an opioid therapeutic agent;
an opioid antagonist;
at least one hydrophobic polymer; and
at least one polycarboxylic acid;
wherein the oral dosage form releases at least 80% of the opioid therapeutic agent after 2 hours of stirring in a 0.1 N HCl solution and 16 hours stirring in a pH 6.8 phosphate buffer solution using a USP Type II paddle apparatus at 75 rpm and 37° C., and wherein the oral dosage form releases less than 40% of the hydromorphone and/or pharmaceutically acceptable salts of hydromorphone after 5 minutes of shaking at 240 cycles/min in a 0.1 N HCl solution and 3 hours of shaking on an orbital shaker at 240 cycles/min in an aqueous solution of 40% ethanol at 25° C., and
wherein the opioid antagonist is sequestered from the opioid therapeutic agent such that the opioid antagonist has no substantial effect on the activity of the opioid therapeutic agent when the dosage form is taken orally as prescribed, but the opioid antagonist is released in an amount that reduces the effectiveness of the opioid therapeutic agent contained in the dosage form when the dosage form is crushed.

2. The oral dosage form of claim 1, wherein the oral dosage form releases between about 10% and about 50% of the opioid therapeutic agent after 2 hours of stirring in a 0.1 N HCl solution and 1 hour stirring in a pH 6.8 phosphate buffer solution using a USP Type II paddle apparatus at 75 rpm and 37° C.

3. The oral dosage form of claim 1, wherein the oral dosage form releases between about 40% and about 70% of the opioid therapeutic agent after 2 hours of stirring in a 0.1 N HCl solution and 10 hours stirring in a pH 6.8 phosphate buffer solution using a USP Type II paddle apparatus at 75 rpm and 37° C.

4. The oral dosage form of claim 1, wherein the at least one hydrophobic polymer comprises at least 20% by weight of the oral dosage form.

5. The oral dosage form of claim 1, wherein the at least one hydrophobic polymer comprises an acrylic acid based polymer and/or a methacrylic acid based polymer.

6. The oral dosage form of claim 1, wherein the at least one hydrophobic polymer comprises an alkyl cellulose.

7. The oral dosage form of claim 1, further comprising one or more hydrophilic polymers.

8. The oral dosage form of claim 1, further comprising one or more hydroxyalkyl celluloses.

9. The oral dosage form of claim 1, further comprising one or more plasticizers.

10. (canceled)

11. The oral dosage form of claim 1, wherein the at least one polycarboxylic acid comprises an α-hydroxy polycarboxylic acid.

12. The oral dosage form of claim 1, wherein the at least one polycarboxylic acid comprises citric acid.

13. (canceled)

14. The oral dosage form of claim 1, further comprising one or more pore formers.

15. (canceled)

16. (canceled)

17. (canceled)

18. (canceled)

19. The oral dosage form of claim 1, wherein the oral dosage form has a hardness of at least about 50 kp.

20. The oral dosage form of claim 1, wherein the oral dosage form has a diameter of greater than about 5 mm.

21. (canceled)

22. (canceled)

23. The oral dosage form of claim 1, wherein the oral dosage form has a moisture content of less than about 5%.

24. The oral dosage form of claim 1, wherein the oral dosage form is disposed in a gelatin-capsule or coated with a gelatin coating.

25. (canceled)

26. The oral dosage form of claim 1, wherein the oral dosage form is not in the form of an aggregate or composite of individual solid particulates.

27. The oral dosage form of claim 1, wherein the oral dosage form is not in the form of a compressed tablet.

28. The oral dosage form of claim 1, wherein the oral dosage form is abuse deterrent.

29. The oral dosage form of claim 1, wherein the oral dosage form is substantially free of digestible C₈-C₅₀ substituted and unsubstituted hydrocarbons.

30. The oral dosage form of claim 1, wherein the oral dosage form is substantially free of C₈-C₅₀ fatty acids, C₈-C₅₀ fatty alcohols, glyceryl esters of C₈-C₅₀ fatty acids, mineral oils, vegetable oils and waxes.

31. The oral dosage form of claim 1, wherein the opioid therapeutic agent is substantially uniformly dispersed within the oral dosage form.

32. A method of providing an opioid therapeutic agent to a patient comprising providing the patient with a monolithic solidified oral dosage form prepared by a thermal process, the oral dosage form comprising the opioid therapeutic agent, an opioid antagonist, and a hydrophobic matrix material wherein the oral dosage form releases at least 80% of the opioid therapeutic agent after 2 hours of stirring in a 0.1 N HCl solution and 16 hours stirring in a pH 6.8 phosphate buffer solution using a USP Type II paddle apparatus at 75 rpm and 37° C., and wherein the oral dosage form releases less than 40% of the hydromorphone and/or pharmaceutically acceptable salts of hydromorphone after 5 minutes of shaking at 240 cycles/min in a 0.1 N HCl solution and 3 hours of shaking on an orbital shaker at 240 cycles/min in an aqueous solution of 40% ethanol at 25° C.; and
wherein the opioid antagonist is sequestered from the opioid therapeutic agent such that the opioid antagonist has no substantial effect on the activity of the opioid therapeutic agent when the dosage form is taken orally as prescribed, but the opioid antagonist is released in an amount that reduces the effectiveness of the opioid therapeutic agent contained in the dosage form when the dosage form is crushed.

33-63. (canceled)

64. A method of formulating a monolithic solidified oral dosage form, comprising:
forming a mixture of hydrophobic matrix material with an opioid therapeutic agent, and an opioid antagonist;
melting at least a portion of the hydrophobic matrix material of the mixture;
permitting the mixture to solidify, wherein the solidified oral dosage releases at least 80% of the opioid therapeutic agent after 2 hours of stirring in a 0.1 N HCl solution and 16 hours stirring in a pH 6.8 phosphate buffer solution using a USP Type II paddle apparatus at 75 rpm and 37° C., and wherein the oral dosage form releases less than 40% of the hydromorphone and/or pharmaceutically acceptable salts of hydromorphone after 5 minutes of shaking at 240 cycles/min in a 0.1 N HCl solution and 3 hours of shaking on an orbital shaker at 240 cycles/min in an aqueous solution of 40% ethanol at 25° C.; and wherein the opioid antagonist is sequestered from the opioid therapeutic agent such that the opioid antagonist has no substantial effect on the activity of the opioid therapeutic agent when the dosage form is taken orally as prescribed, but the opioid antagonist is released in an amount that reduces the effectiveness of the opioid therapeutic agent contained in the dosage form when the dosage form is crushed.

65-98. (canceled)
